(19) United States
(12) Patent Application Publication
(10) Pub. No.: US 2007/0027321 A1
(43) Pub. Date: Feb. 1, 2007

(54) Bipyridyl amines and ethers as modulators of metabotropic glutamate receptor-5

(76) Inventors: Theodore M. Kamenecka, San Diego, CA (US); Jean-Michel Vernier, San Diego, CA (US); Celine Bonnefous, San Diego, CA (US); Steven P. Govek, San Diego, CA (US); John H. Hutchinson, La Jolla, CA (US)

Correspondence Address: MERCK AND CO., INC, P O BOX 2000, RAHWAY, NJ, 07065-0907, US

(21) Appl. No.: 10/570,068

(22) PCT Filed: Aug. 27, 2004
(86) PCT No.: PCT/US04/27916

§ 371 Date: Mar. 1, 2006

Related U.S. Application Data
(60) Provisional application No. 60/499,627, filed on Sep. 2, 2003.

(51) Int. Cl. A61K 31/4743; C07D 471/02; C07D 401/02; C07D 417/14

(52) U.S. Cl. 546/114; 546/264; 546/261; 546/256

(57) ABSTRACT

The present invention is directed to novel bipyridyl amine and ether compounds such as those of Formula (I): (I) (where R?1#191, R?2#191, R?3#191, X and Y are as defined herein) which are mGluR5 modulators useful in the treatment or prevention of diseases and conditions in which mGluR5 is involved, including but not limited to psychiatric and mood disorders such as schizophrenia, anxiety, depression, bipolar disorders, and panic, as well as in the treatment of pain, Parkinson's disease, cognitive dysfunction, epilepsy, circadian rhythm and sleep disorders, such as shift-work induced sleep disorder and jet-lag, drug addiction, drug abuse, drug withdrawal, obesity and other diseases. The invention is also directed to pharmaceutical compositions comprising these compounds. This invention further provides a method of treatment of these disorders and conditions by the administration of an effective amount of these novel bipyridyl amine and/or ether compounds and/or compositions containing these compounds.

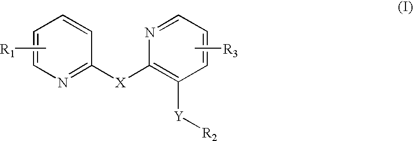

### BACKGROUND OF THE INVENTION

A major excitatory neurotransmitter in the mammalian nervous system is the glutamate molecule, which binds to neurons, thereby activating cell surface receptors. Such surface receptors are characterized as either ionotropic or metabotropic glutamate receptors. The metabotropic glutamate receptors (“mGluR”) are G protein-coupled receptors that activate intracellular second messenger systems when bound to glutamate. Activation of mGluR results in a variety of cellular responses. In particular, mGluR1 and mluR5 activate phospholipase C, which is followed by mobilizing intracellular calcium.

Modulation of metabotropic glutamate receptor subtype 5 (mGluR5) is useful in the treatment of diseases that affect the nervous system (see for example W. P. J. M Spooren et al., Trends Pharmacol. Sci., 22:331-337 (2001) and references cited therein). For example, recent evidence demonstrates the involvement of mGluR5 in nociceptive processes and that modulation of mGluR5 using mGluR5-selective compounds is useful in the treatment of various pain states, including acute, persistent and chronic pain [K Walker et al., Neuropharmacology, 40:1-9 (2001); F. Bordi, A. Ugolini Brain Res., 871:223-233 (2001)], inflammatory pain [K Walker et al., Neuropharmacology, 40: 10-19 (2001); Bhave et al. Nature Neurosci. 4:417-423 (2001)] and neuropathic pain [Dogmil et al. Neurosci. Lett. 292:115-118 (2000)].

Further evidence supports the use of modulators of mGluR5 in the treatment of psychiatric and neurological disorders. For example, mGluR5-selective compounds such as 2-methyl-6-phenylethynyl)-pyridine (“MPEP”) are effective in animal models of mood disorders, including anxiety and depression [W. P. J. M Spooren et al., J. Pharmacol. Exp. Ther., 295:1267-1275 (2000); E. Tatarczynska et al, Brit. J. Pharmacol., 132:1423-1430 (2001); A. Klodzynska et al, Pol. J. Pharmacol., 132:1423-1430 (2001)]. Gene expression data from humans indicate that modulation of mGluR5 may be useful for the treatment of schizophrenia [T. Ohnuma et al, Mol. Brain. Res., 56:207-217 (1998); ibid, Mol. Brain. Res., 85:2431 (2000)]. Studies have also shown a role for mGluR5, and the potential utility of mGluR5-modulatory compounds, in the treatment of movement disorders such as Parkinson's disease [W. P. J. M Spooren et al., Europ. J. Pharmacol. 406:403-410 (2000); H. Awad et al., J. Neurosci. 20:7871-7879 (2000); K. Ossawa et al. Neuropharmacol. 41:413-420 (2001)]. Other research supports a role for mGluR5 modulation in the treatment of cognitive dysfunction [G. Riedel et al, Neuropharmacol. 39:1943-1951 (2000)], epilepsy [A. Chapman et al, Neuropharmacol. 39:1567-1574 (2000)] and neuroprotection [V. Bruno et al, Neuropharmacol. 39:2223-2230 (2000)]. Studies with mGluR5 knockout mice and MPEP also suggest that modulation of these receptors may be useful in the treatment of drug addiction, drug abuse and drug withdrawal [C. Chiamulera et al. Nature Neurosci. 4:873-874 (2001)].

International Patent Publications WO 01/12627 and WO 99/26927 describe heteropolycyclic compounds and their use as metabotropic glutamate receptor antagonists.

U.S. Pat. No. 3,647,809 describes pyridyl-1,2,4-oxadiazole derivatives. U.S. Pat. No. 4,022,901 describes 3-pyridyl-5-isothiocyanophenyl oxadiazoles. International Patent Publication WO 98/17652 describes oxadiazoles, WO 97/03967 describes various substituted aromatic compounds, JP 13233767A and WO 94/22846 describe various heterocyclic compounds.

Compounds that include ringed systems are described by various investigators as effective for a variety of therapies and utilities. For example, International Patent Publication No. WO 98/25883 describes ketobenzamides as calpain inhibitors, European Patent Publication No. EP 811610 and U.S. Pat. Nos. 5,679,712, 5,693,672 and 5,747,541 describe substituted benzoylguanidine sodium channel blockers, and U.S. Pat. No. 5,736,297 describes ring systems useful as a photosensitive composition.

However, there remains a need for novel compounds and compositions that therapeutically inhibit mGluR5 with minimal side effects.

### SUMMARY OF THE INVENTION

The present invention is directed to novel bipyridyl amine and ether compounds such as those of Formula (I): 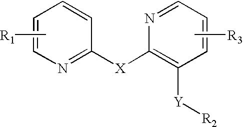
(where R₁, R₂, R₃, X and Y are as defined herein) which are mGluR5 modulators useful in the treatment or prevention of diseases and conditions in which mGluR5 is involved, including but not limited to psychiatric and mood disorders such as schizophrenia, anxiety, depression, bipolar disorders, and panic, as well as in the treatment of pain, Parkinson's disease, cognitive dysfunction, epilepsy, circadian rhythm and sleep disorders, such as shift-work induced sleep disorder and jet-lag, drug addiction, drug abuse, drug withdrawal, obesity and other diseases. The invention is also directed to pharmaceutical compositions comprising these compounds. This invention further provides a method of treatment of these disorders and conditions by the administration of an effective amount of these novel bipyridyl amine and/or ether compounds and/or compositions containing these compounds.

### DETAILED DESCRIPTION OF THE INVENTION

The present invention provides novel compounds of formula I: 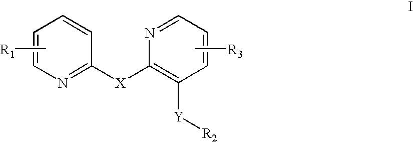
or a pharmaceutically acceptable salt thereof wherein:
R₁ is selected from:

1) hydrogen,

2) C₁₋₁₀alkyl,

3) C₂₋₁₀alkenyl,

4) C₂₋₁₀alkynyl

5) cycloalkyl,

6) heterocyclyl,

7) aryl,

8) heteroaryl,

—NR^{d}R^{e},

—CO₂R^{d},

—OR^{d},

—CN, and

halogen,

where alkyl, alkenyl, alkynyl, cycloalkyl and heterocyclyl are optionally substituted with one to four substituents selected from R^{a}, and where aryl and heteroaryl are optionally substituted with one to four substituents independently selected from R^{b};

R₂ is selected from:

1) hydrogen,

2) C₁₋₁₀alkyl,

3) C₂₋₁₀alkenyl,

C₂₋₁₀alkynyl,

cycloalkyl,

heterocyclyl,

aryl, and

heteroaryl,

where alkyl, alkenyl and alkynyl, cycloalkyl and heterocyclyl, aryl, and heteroaryl are optionally substituted with one to four substituents independently selected from R^{b};

R₃ is selected from:

1) R^{b},

2) hydrogen,

3)-Z-aryl,

-Z-heteroaryl,

where aryl and heteroaryl are optionally substituted with one to four substituents independently selected from R^{b}, and where Z is a bond, C, O, S or NR^{d};

R^{a} is selected from:

1) hydrogen,

2) —OR^{d},

3) —NO₂,

4) halogen,

5) —S(O)ₘR^{d},

6) —SR^{d},

7) —S(O)ₘNR^{d}R^{e},

8) —NR^{d}R^{e},

9) —C(O)R^{d},

10) —CO₂R^{d},

11) —OC(O)R^{d},

12) —CN,

13) —C(O)NR^{d}R^{e},

14) —NR^{d}C(O)R^{e},

15) —OC(O)NR^{d}R^{e},

16) —NR^{d}C(O)OR^{e},

17) —NR^{d}C(O)NR^{d}R^{e},

18) —CR^{d}(N—OR^{d}),

19) CF₃, and

20) —OCF₃;

R^{b} is selected from:

1) R^{a},

2) C₁₋₁₀ alkyl,

3) C₂₋₁₀ alkenyl,

4) C₂₋₁₀ alkynyl,

5) cycloalkyl,

6) heterocyclyl,

7) aryl, and

8) heteroaryl,

where alkyl, alkenyl, alkynyl, cycloalkyl, heterocyclyl, aryl, heteroaryl are optionally substituted with one to four substituents selected from a group independently selected from R^{c};

R^{c} is selected from:

1) halogen,

2) amino,

3) carboxy,

4) cyano,

5) C₁₋₄alkyl,

6) C₁₋₄alkoxy,

7) aryl,

8) aryl C₁₋₄alkyl,

9) heteroaryl,

10) hydroxy,

11) CF₃, and

12) aryloxy;

R^{d} and R^{e} are independently selected from hydrogen, C₁₋₁₀alkyl, C₂₋₁₀alkenyl, C₂₋₁₀alkynyl and Cy, where alkyl, alkenyl, alkynyl and Cy are optionally substituted with one to four substituents independently selected from R^{c};

or R^{d} and R^{e} together with the atoms to which they are attached form a ring of 4 to 7 members containing 0-2 additional heteroatoms independently selected from oxygen, sulfur and nitrogen;

Cy is independently selected from cycloalkyl, heterocyclyl, aryl, or heteroaryl;

m is 1 or 2;

X is —NR^{d}—, —O—, or —S—; and

Y is a bond, —O—, —NR^{a}— or —S—.

An additional embodiment of the invention includes compounds of formula I, or a pharmaceutically acceptable salt thereof wherein:

R₁ is C₁₋₁₀allyl, optionally substituted with one to four substituents selected from R^{a},

R₂ is C₁₋₁₀alkyl, optionally substituted with one to four substituents independently selected from R^{b};

R₃, R^{a}, R^{b}, R^{c}, R^{d}, R^{e} and m are as described above;

X is —NR^{d}—; and

Y is —O—.

As used herein, “alkyl” as well as other groups having the prefix “alk” such as, for example, alkoxy, alkanoyl, alkenyl, alkynyl and the like, means carbon chains which may be linear or branched or combinations thereof. Examples of alkyl groups include methyl, ethyl, propyl, isopropyl, butyl, sec- and tert-butyl, pentyl, hexyl, heptyl and the like. “Alkenyl”, “alkynyl” and other like terms include carbon chains containing at least one unsaturated C—C bond.

The term “C₀₋₁₀alkyl” includes alkyls containing 10, 9, 8, 7, 6, 5, 4, 3, 2, 1, or no carbon atoms. An alkyl with no carbon atoms, i.e., C₀, is a hydrogen atom substituent when the alkyl is a terminal group and is a direct bond when the alkyl is a bridging group.

The term “cycloalkyl” means carbocycles containing no heteroatoms, and includes mono-, bi- and tricyclic saturated carbocycles, as well as fused ring systems. Such fused ring systems can include one ring that is partially or fully unsaturated such as a benzene ring to form fused ring systems such as benzofused carbocycles. Cycloalkyl includes such fused ring systems as spirofused ring systems. Examples of cycloalkyl include cyclopropyl, cyclobutyl, cyclopentyl, cyclohexyl, decahydronaphthalene, adamantane, indanyl, indenyl, fluorenyl, 1,2,3,4-tetrahydronaphalene and the like. Similarly, “cycloalkenyl” means carbocycles containing no heteroatoms and at least one non-aromatic C—C double bond, and include mono-, bi- and tricyclic partially saturated carbocycles, as well as benzofused cycloalkenes. Examples of cycloalkenyl include cyclohexenyl, indenyl, and the like. Collectively, cycloalkyls and cycloalkenyls are known as “cyclyls”

The term “aryl” means an aromatic substituent which is a single ring or multiple rings fused together. When formed of multiple rings, at least one of the constituent rings is aromatic. Possible aryl substituents include phenyl and naphthyl groups.

The term “cycloalkyloxy” unless specifically stated otherwise includes a cycloalkyl group connected by a short C₁₋₂alkyl length to the oxy connecting atom.

The term “hetero” unless specifically stated otherwise includes one or more O, S, or N atoms. For example, heterocycloalkyl and heteroaryl include ring systems that contain one or more O, S, or N atoms in the ring, including mixtures of such atoms. The hetero atoms replace ring carbon atoms. Thus, for example, a heterocycloC₅alkyl is a five-member ring containing from 4 to no carbon atoms. Examples of heteroaryls include pyridinyl, quinolinyl, isoquinolinyl, pyridazinyl, pyrimidinyl, pyrazinyl, quinoxalinyl, furyl, benzofuryl, dibenzofuryl, thienyl, benzthienyl, pyrrolyl, indolyl, pyrazolyl, indazolyl, oxazolyl, benzoxazolyl, isoxazolyl, thiazolyl, benzothiazolyl, isothiazolyl, imidazolyl, benzimidazolyl, oxadiazolyl, thiadiazolyl, triazolyl, and tetrazolyl. Examples of heterocycloalkyls include azetidinyl, pyrrolidinyl, piperidinyl, piperazinyl, morpholinyl, tetrahydrofuranyl, imidazolinyl, pyrolidin-2-one, piperidin-2-one, and thiomorpholinyl.

Similarly, the term “heteroC₀₋₄alkyl” means a heteroalkyl containing 3, 2, 1, or no carbon atoms. However, at least one heteroatom must be present. Thus, as an example, a heteroC₀₋₄alkyl having no carbon atoms but one N atom would be a —NH— if a bridging group and a —NH₂ if a terminal group. Analogous bridging or terminal groups are clear for an O or S heteroatom.

The term “amine” unless specifically stated otherwise includes primary, secondary and tertiary amines substituted with C₀₋₆alkyl.

The term “carbonyl” unless specifically stated otherwise includes a C₀₋₆alkyl substituent group when the carbonyl is terminal.

The term “halogen” includes fluorine, chlorine, bromine and iodine atoms.

The term “optionally substituted” is intended to include both substituted and unsubstituted. Thus, for example, optionally substituted aryl could represent a pentafluorophenyl or a phenyl ring. Further, optionally substituted multiple moieties such as, for example, alkylaryl are intended to mean that the aryl and the aryl groups are optionally substituted. If only one of the multiple moieties is optionally substituted then it will be specifically recited such as “an alkylaryl, the aryl optionally substituted with halogen or hydroxyl.”

Compounds described herein contain one or more double bonds and may thus give rise to cis/trans isomers as well as other conformational isomers. The present invention includes all such possible isomers as well as mixtures of such isomers.

Compounds described herein can contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof. The above Formula I is shown without a definitive stereochemistry at certain positions. The present invention includes all stereoisomers of Formula I and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

The independent syntheses of these diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Alternatively, any enantiomer of a compound may be obtained by stereoselective synthesis using optically pure starting materials or reagents of known configuration by methods well known in the art.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. In certain embodiments of the invention said salts are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. In some embodiments the corresponding salts are citric, hydrobromic, hydrochloric, maleic, phosphoric, sulfuric, and tartaric acids.

The pharmaceutical compositions of the present invention comprise a compound represented by Formula I (or pharmaceutically acceptable salts thereof) as an active ingredient, a pharmaceutically acceptable carrier and optionally other therapeutic ingredients or adjuvants. Such additional therapeutic ingredients include, for example, i) opiate agonists or antagonists, ii) calcium channel antagonists, iii) 5HT receptor agonists or antagonists iv) sodium channel antagonists, v) NMDA receptor agonists or antagonists, vi) COX-2 selective inhibitors, vii) NK1 antagonists, viii) non-steroidal anti-inflammatory drugs (“NSAID”), ix) GABA-A receptor modulators, x) dopamine agonists or antagonists, xi) selective serotonin reuptake inhibitors (“SSRI”) and/or selective serotonin and norepinephrine reuptake inhibitors (“SSNRI”), xii) tricyclic antidepressant drugs, xiv) norepinephrine modulators, xv) L-DOPA, xvi) buspirone, xvii) lithium, xviii) valproate, ixx) neurontin (gabapentin), xx) olanzapine, xxi) nicotinic agonists or antagonists including nicotine, xxii) muscarinic agonists or antagonists, xxiii) heroin substituting drugs such as methadone, levo-alpha-acetylmethadol, buprenorphine and naltrexone, and xxiv) disulfiram and acamprosate. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

The compounds of the present invention may be used in combination with one or more other drugs in the treatment, prevention, control, amelioration, or reduction of risk of diseases or conditions for which compounds of Formula I or the other drugs may have utility, where the combination of the drugs together are safer or more effective than either drug alone. Such other drug(s) may be administered, by a route and in an amount commonly used therefor, contemporaneously or sequentially with a compound of Formula I. When a compound of Formula I is used contemporaneously with one or more other drugs, a pharmaceutical composition in unit dosage form containing such other drugs and the compound of Formula I may be employed. However, the combination therapy may also include therapies in which the compound of Formula I and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of Formula L

The above combinations include combinations of a compound of the present invention not only with one other active compound, but also with two or more other active compounds. Likewise, compounds of the present invention may be used in combination with other drugs that are used in the prevention, treatment, control, amelioration, or reduction of risk of the diseases or conditions for which compounds of the present invention are useful. Such other drugs may be administered, by a route and in an amount commonly used therefor, contemporaneously or sequentially with a compound of the present invention. When a compound of the present invention is used contemporaneously with one or more other drugs, a pharmaceutical composition containing such other drugs in addition to the compound of the present invention may be employed. Accordingly, the pharmaceutical compositions of the present invention include those that also contain one or more other active ingredients, in addition to a compound of the present invention.

Creams, ointments, jellies, solutions, or suspensions containing the compound of Formula I can be employed for topical use. Mouth washes and gargles are included within the scope of topical use for the purposes of this invention.

All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release. Oral tablets may also be formulated for immediate release, such as fast melt tablets or wafers, rapid dissolve tablets or fast dissolve films.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Dosage levels from about 0.01 mg/kg to about 140 mg/kg of body weight per day are useful in the treatment of psychiatric and mood disorders such as, for example, schizophrenia, anxiety, depression, panic, bipolar disorders, and circadian disorders, as well as being useful in the treatment of pain which are responsive to mGluR5 inhibition, or alternatively about 0.5 mg to about 7 g per patient per day. For example, schizophrenia, anxiety, depression, and panic may be effectively treated by the administration of from about 0.01 mg to 75 mg of the compound per kilogram of body weight per day, or alternatively about 0.5 mg to about 3.5 g per patient per day. Pain may be effectively treated by the administration of from about 0.01 mg to 125 mg of the compound per kilogram of body weight per day, or alternatively about 0.5 mg to about 5.5 g per patient per day. Further, it is understood that the mGluR5 inhibiting compounds of this invention can be administered at prophylactically effective dosage levels to prevent the above-recited conditions.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The amount of active ingredient that may be combined with the carrier materials to produce a single dosage form will vary depending upon the host treated and the particular mode of administration. For example, a formulation intended for the oral administration to humans may conveniently contain from about 0.5 mg to about 5 g of active agent, compounded with an appropriate and convenient amount of carrier material which may vary from about 5 to about 95 percent of the total composition. Unit dosage forms will generally contain between from about 1 mg to about 1000 mg of the active ingredient, typically 25 mg, 50 mg, 100 mg, 200 mg, 300 mg, 400 mg, 500 mg, 600 mg, 800 mg or 1000 mg.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

In practice, the compounds represented by Formula I, or pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compound represented by Formula I, or pharmaceutically acceptable salts thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound or a pharmaceutically acceptable salt of Formula I. The compounds of Formula I, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the typical oral dosage units whereby solid pharmaceutical carriers are employed. Optionally, tablets may be coated by standard aqueous or nonaqueous techniques

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet may contain from about 0.1 mg to about 500 mg of the active ingredient and each cachet or capsule may contain from about 0.1 mg to about 500 mg of the active ingredient. Thus, a tablet, cachet, or capsule conveniently contains 0.1 mg, 1 mg, 5 mg, 25 mg, 50 mg, 100 mg, 200 mg, 300 mg, 400 mg, or 500 mg of the active ingredient taken one or two tablets, cachets, or capsules, once, twice, or three times daily.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, may be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound represented by Formula I of this invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. The mixture may form unit dose suppositories. Suitable carriers include cocoa butter and other materials commonly used in the art. The suppositories may be conveniently formed by first admixing the composition with the softened or melted carrier(s) followed by chilling and shaping in moulds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound described by Formula I, or pharmaceutically acceptable salts thereof, may also be prepared in powder or liquid concentrate form.

The compounds and pharmaceutical compositions of this invention have been found to exhibit biological activity as mGluR5 inhibitors. Accordingly, another aspect of the invention is the treatment in mammals of, for example, schizophrenia, anxiety (including panic, agoraphobia or other specific phobias, obsessive-compulsive disorders, post-traumatic stress disorders, acute stress disorder, generalized anxiety disorder, eating disorders, substance-induced anxiety disorders, non-specific anxiety disorders), depression, bipolar disorders, dementia, psychosis, circadian rhythm and sleep disorders, pain (including acute pain, persistent pain, chronic pain, inflammatory pain or neuropathic pain), Parkinson's disease, Alzheimer's disease, cognitive dysfunction, epilepsy, obesity, drug addiction, drug abuse and drug withdrawal (including tobacco withdrawal)—maladies that are amenable to amelioration through inhibition of mGluR5—by the administration of an effective amount of the compounds of this invention. The term “mammals” includes humans, as well as other animals such as, for example, dogs, cats, horses, pigs, and cattle. Accordingly, it is understood that the treatment of mammals other than humans is the treatment of clinical correlating afflictions to those above recited examples that are human afflictions.

Further, as described above, the compound of this invention can be utilized in combination with other therapeutic compounds. In particular, the combinations of the mGluR5 inhibiting compound of this invention can be advantageously used in combination with i) opiate agonists or antagonists, ii) calcium channel antagonists, iii) 5HT receptor agonists or antagonists iv) sodium channel antagonists, v) NMDA receptor agonists or antagonists, vi) COX-2 selective inhibitors, vii) NK1 antagonists, viii) non-steroidal anti-inflammatory drugs (“NSAID”), ix) GABA-A receptor modulators, x) dopamine agonists or antagonists, xi) selective serotonin reuptake inhibitors (“SSRI”) and/or selective serotonin and norepinephrine reuptake inhibitors (“SSNRI”), xii) tricyclic antidepressant drugs, xiii) norepinephrine modulators, xiv) L-DOPA, xv) buspirone, xvi) lithium, xvii) valproate, xviii) neurontin (gabapentin), xix) olanzapine, xx) nicotinic agonists or antagonists including nicotine, xxi) muscarinic agonists or antagonists, xxii) heroin substituting drugs such as methadone, levo-alpha-acetylmethadol, buprenorphine and naltrexone, and xxiii) disulfiram and acamprosate.

The weight ratio of the compound of the compound of the present invention to the other active ingredient(s) may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, or from about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s), and via the same or different routes of administration.

The subject compounds are useful in a method of modulating mGluR5 in a patient such as a mammal in need of such antagonism comprising the administration of an effective amount of the compound. The present invention is directed to the use of the compounds disclosed herein as modulators of mGluR5. In addition to primates, especially humans, a variety of other mammals can be treated according to the method of the present invention.

Another embodiment of the present invention is directed to a method for the treatment, control, amelioration, or reduction of risk of a disease or disorder in which mGluR5 is involved in a patient that comprises administering to the patient a therapeutically effective amount of a compound that is a modulator of mGluR5.

The present invention is further directed to a method for the manufacture of a medicament for modulation of mGluR5receptors activity in humans and animals comprising combining a compound of the present invention with a pharmaceutical carrier or diluent.

The term “therapeutically effective amount” means the amount of the subject compound that will elicit the biological or medical response of a tissue, system, animal or human that is being sought by the researcher, veterinarian, medical doctor or other clinician. As used herein, the term “treatment” refers both to the treatment and to the prevention or prophylactic therapy of the mentioned conditions, particularly in a patient who is predisposed to such disease or disorder.

The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention or a prodrug of a compound of the invention to the individual in need of treatment.

The ability of the compounds of the present invention to act as mGluR5 modulators makes them useful pharmacological agents for disorders that involve mGluR5 in humans and animals, but particularly in humans.

The subject compounds are further useful in a method for the prevention, treatment, control, amelioration, or reduction of risk of the aforementioned diseases, disorders and conditions in combination with other agents.

### Assays Demonstrating Biological Activity

The compounds of this invention were tested against the hmGluR5a receptor stably expressed in mouse fibroblast Ltk⁻ cells (the hmGluR5a/L38-20 cell line) and activity was detected by changes in [Ca⁺⁺]ᵢ, measured using the fluorescent Ca⁺⁺-sensitive dye, fura-2. InsP assays were performed in mouse fibroblast Ltk⁻ cells LM5a cell line) stably expressing hmGluR5a. The assays described in International Patent Publication WO 0116121 can be used.

Calcium Flux Assay

The activity of compounds was examined against the hmGluR5a receptor stably expressed in human embryonic kidney HEK293 cells (the hmGluR5a cell line designated hm5a). See generally Daggett et al., Neuropharmacology 34:871-886 (1995). Receptor activity was detected by changes in intracellular calcium ([Ca²⁺ ]ᵢ) measured using the fluorescent calcium-sensitive dye, fura-2. The hm5a cells were plated onto 96-well plates, and loaded with 3 μM fura-2 for 1 h. Unincorporated dye was washed from the cells, and the cell plate was transferred to a 96-channel fluorimeter (SIBIA-SAIC, La Jolla, Calif.) which is integrated into a fully automated plate handling and liquid delivery system. Cells were excited at 350 and 385 nm with a xenon source combined with optical filters. Emitted light was collected from the sample through a dichroic mirror and a 510 nm interference filter and directed into a cooled CCD camera (Princeton Instruments). Image pairs were captured approximately every is, and ratio images were generated after background subtraction. After a basal reading of 20 s, an EC₈₀ concentration of glutamate (10 μM) was added to the well, and the response evaluated for another 60 s. The glutamate-evoked increase in [Ca′]ᵢ in the presence of the screening compound was compared to the response of glutamate alone (the positive control).

Phosphatidylinositol Hydrolysis (PI) Assays

Inositolphosphate assays were performed as described by Berridge et al. [Berridge et al, Biochem. J. 206: 587-5950 (1982); and Nakajima et al., J. Biol. Chem. 267:2437-2442 (1992)] with slight modifications. Mouse fibroblast Ltk cells expressing hmGluR5 (hmGluR5/L38-20 cells) were seeded in 24-well plates at a density of 8×105 cells/well. One μCi of [³H]-inositol (Amersham PT6-271; Arlington Heights, Ill.; specific activity=17.7 Ci/mmol) was added to each well and incubated for 16 h at 37° C. Cells were washed twice and incubated for 45 min in 0.5 mL of standard Hepes buffered saline buffer (HBS; 125 mM NaCl, 5 mM KCI, 0.62 mM MgSO₄, 1.8 mM CaCl₂, 20 mM HEPES, 6 mM glucose, pH to 7.4). The cells were washed with HBS containing 10 mM LiCl, and 400 μL buffer added to each well. Cells were incubated at 37° C. for 20 min. For testing, 50 μL of 10X compounds used in the practice of the invention (made in HBS/LiCl (100 mM)) was added and incubated for 10 minutes. Cells were activated by the addition of 100 μM glutamate, and the plates left for 1 hour at 37° C. The incubations were terminated by the addition of 1 mL ice-cold methanol to each well. In order to isolate inositol phosphates (IPs), the cells were scraped from wells, and placed in numbered glass test tubes. One mL of chloroform was added to each tube, the tubes were mixed, and the phases separated by centrifugation. IPs were separated on Dowex anion exchange columns (AG 1-X8 100-200 mesh formate form). The upper aqueous layer (750 μL) was added to the Dowex columns, and the columns eluted with 3 mL of distilled water. The eluents were discarded, and the columns were washed with 10 mLs of 60 mM ammonium formate/5 mM Borax, which was also discarded as waste. Finally, the columns were eluted with 4 mL of 800 mM ammonium formate/0.1M formic acid, and the samples collected in scintillation vials. Scintillant was added to each vial, and the vials shaken, and counted in a scintillation counter after 2 hours. Phosphatidylinositol hydrolysis in cells treated with certain exemplary compounds was compared to phosphatidylinositol hydrolysis in cells treated with the agonist alone in the absence of compound.

In general, the compounds of this application have mGluR5 inhibitory activity as shown by IC₅₀ values of less than 10 μM in the calcium flux assay or inhibition at a concentration of 100 μM in the PI assay. The compounds should have IC₅₀ values of less than 1 μM in the calcium flux assay and IC₅₀ values of less than 10 μM in the PI assay. Alternatively, the compounds should have IC₅₀ values of less than 500 nM in the calcium flux assay and IC₅₀ values of less than 1 μM in the PI assay

The compounds described in examples 1 to 55 have mGluR5 inhibitory activity as shown by inhibition at 10 μM or less in the calcium flux assay or 100 μM or less in the PI assay. Many of the compounds show inhibition at 10 μM or less in the calcium flux assay or inhibition at 100 μM or less in the PI assay.

The examples that follow are intended as an illustration of certain embodiments of the invention and no limitation of the invention is implied.

Unless specifically stated otherwise, the experimental procedures were performed under the following conditions. All operations were carried out at room or ambient temperature—that is, at a temperature in the range of 18-25° C. Evaporation of solvent was carried out using a rotary evaporator under reduced pressure (600-4000 pascals: 4.5-30 mm. Hg) with a bath temperature of up to 60° C. The course of reactions was followed by thin layer chromatography (TLC) and reaction times are given for illustration only. The structure and purity of all final products were assured by at least one of the following techniques: TLC, mass spectrometry, nuclear magnetic resonance (NMR) spectrometry or HPLC analysis. When given, yields are for illustration only. When given, NMR data is in the form of delta (δ) values for major diagnostic protons, given in parts per million (ppm) relative to tetramethylsilane (TMS) as internal standard, determined at 500 MHz using the indicated solvent. Conventional abbreviations used for signal shape are: s. singlet; d. doublet; t. triplet; m. multiplet; br. broad; etc. Chemical symbols have their usual meanings; the following abbreviations are used: v (volume), w (weight), b.p. (boiling point), m.p. (melting point), L (liter(s)), mL (milliliters), g (gram(s)), mg (milligrams(s)), mol (moles), mmol (millimoles), eq (equivalent(s)).

### Methods of Synthesis

Compounds of the present invention can be prepared according to the following methods. The substituents are the same as in Formula (I) except where defined otherwise, or apparent to one in the art.

In the below-described Scheme, R, R₁, R₂, R₃, X and Y are as defined above. Other variables are understood by one in the art by the context in which they are used.

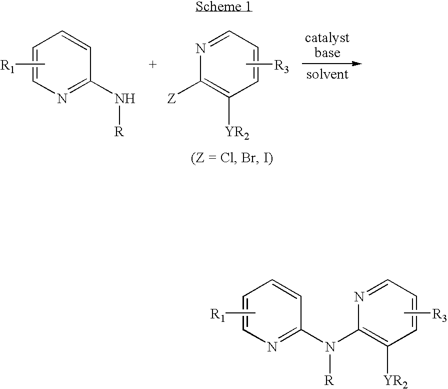

Thus, in Scheme 1, a suitably substituted pyridine containing a halogen atom Z (Cl, Br, or I) may be coupled with an appropriately funtionalized 2-aminopyridine in the presence of a stoichiometric or catalytic amount of a palladium catalyst such as Pd(Ph₃P)₄, PdCl₂(Ph₃P)₂, Pd₂ dba₃, Pd(OAc)₂, PdCl₂dppf and the like. Typically a base (e.g. K₂CO₃, Cs₂CO₃, K₃PO₄, Et₃N, NaOtBu, KOtBu, etc . . . ) will also be present and the reaction carried out in a suitable solvent (DCM, THF, DME, DMF, DMAC, CH₃CN, dioxane, toluene, benzene, etc. . . .). Additionally, ligands such as BINAP, di-tert-butyl phosphinobiphenyl, di-cyclohexylphosphino biphenyl, tri tert-butylphosphine, XANTPHOS, triphenylarsine and the like may be added. The reaction is conducted under an inert atmosphere (N₂ or argon) at a temperature between 50-120 C. The reaction mixture is then maintained at a suitable temperature for a time in the range of about 2 up to 48 h with 12 h typically being sufficient (see for example Yang, B. H.; Buchwald, S. L. J. Organomet. Chem. 1999, 576, 12546 and Wolfe, J. P.; Tomori, H.; Sadighi, J. P.; Yin, J.; Buchwald, S. L. J. Org. Chem. 2000, 65, 1158-1174). Alternatively, the reaction may be carried out under microwave irradiation in a sealed tube. These reactions are typically conducted at a temperature between 110-180 C for a time range of 5 min to 2 h with 20 min typically being sufficient. The product from the reaction can be isolated and purified employing standard techniques, such as solvent extraction, chromatography, crystallization, distillation and the like.

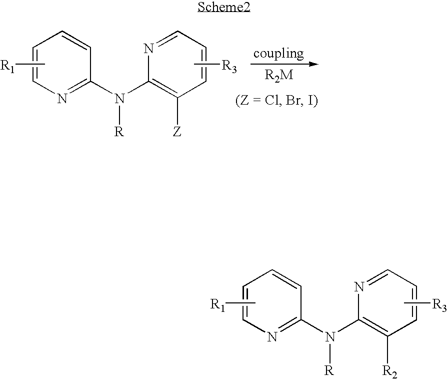

Another embodiment of the present invention is illustrated in Scheme 2. This biarylamine product may then be coupled with an R₂-group under metal-catalyzed cross-coupling conditions where M is a metallic or metalloid species such as B(OR)₂, Li, MgHal, SnR3, ZnHal, SiR₃ and the like which is capable of undergoing a metal-catalyzed cross-coupling reaction. The coupling may be promoted by a homogeneous catalyst such as Pd(PPh₃)₄, or by a heterogeneous catalyst such as Pd on carbon in a suitable solvent (e.g. THF, DME, toluene, MeCN, DMF, H₂O etc.). Typically a base, such as K₂CO₃, NEt₃, and the like, will also be present in the reaction mixture. Other promoters may also be used such as CsF. The reaction mixture is maintained at rt, or heated to a temperature between 30° C. to 150° C. The reaction mixture is then maintained at a suitable temperature for a time in the range of about 4 up to 48 h, with about 18 h typically being sufficient (see for example Miyaura, N.; Suzuki, A. Chem. Rev. 1995, 95, 2457-2483). Alternatively, the reaction may be carried out under microwave irradiation in a sealed tube. These reactions are typically conducted at a temperature between 110-180 C for a time range of 5 min to 2 h with 20 min typically being sufficient. The product from the reaction can be isolated and purified employing standard techniques, such as solvent extraction, chromatography, crystallization, distillation and the like.

Another embodiment of the present invention is illustrated in Scheme 3. Thus, as suitably substituted 2-halopyridine is coupled to an appropriately funtionalized 2-aminopyridine in the presence of a stoichiometric or catalytic amount of a palladium catalyst such as Pd(Ph₃P)₄, PdCl₂ (Ph₃P)₂, Pd₂ dba₃, Pd(OAc)₂, PdCl₂dppf and the like. Typically a base (e.g. K₂CO₃, Cs₂CO₃, K₃PO₄, Et₃N, NaOtBu, KOtBu, etc . . . ) will also be present and the reaction carried out in a suitable solvent (DCM, THF, DME, DMF, DMAC, CH₃CN, dioxane, toluene, benzene, etc. . . .). Additionally, ligands such as BINAP, di-tert-butyl phosphinobiphenyl, di-cyclohexylphosphino biphenyl, tri tert-butylphosphine, XANTPHOS, triphenylarsine and the like may be added. The reaction is conducted under an inert atmosphere (N₂ or argon) at a temperature between 50-120 C. The reaction mixture is then maintained at a suitable temperature for a time in the range of about 2 up to 48 h with 12 h typically being sufficient. Alternatively, the reaction may be carried out under microwave irradiation in a sealed tube. These reactions are typically conducted at a temperature between 110-180 C for a time range of 5 min to 2 h with 20 min typically being sufficient. The product from the reaction can be isolated and purified employing standard techniques, such as solvent extraction, chromatography, crystallization, distillation and the like.

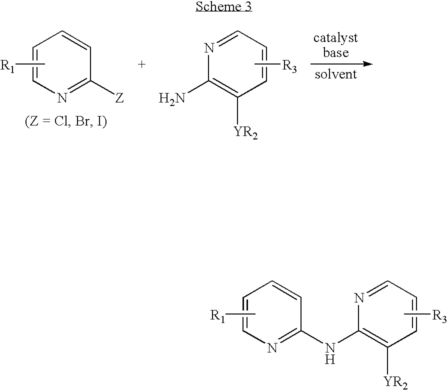

Another embodiment of the present invention is illustrated in Scheme 4 where Z is a halogen atom. This biarylamine product may then be coupled with an R₃-group under metal-catalyzed cross-coupling conditions where M is a metallic or metalloid species such as B(OR)₂, Li, MgHal, SnR₃, ZnHal, SiR₃ and the like which is capable of undergoing a metal-catalyzed cross-coupling reaction. The coupling may be promoted by a homogeneous catalyst such as Pd(PPh₃)₄, or by a heterogeneous catalyst such as Pd on carbon in a suitable solvent (e.g. THF, DME, toluene, MeCN, DMF, H₂O etc.). Typically a base, such as K₂CO₃, NEt₃, and the like, will also be present in the reaction mixture. Other promoters may also be used such as CsF. The reaction mixture is maintained at rt, or heated to a temperature between 30° C. to 150° C. The reaction mixture is then maintained at a suitable temperature for a time in the range of about 4 up to 48 h, with about 18 h typically being sufficient. Alternatively, the reaction may be carried out under microwave irradiation in a sealed tube. These reactions are typically conducted at a temperature between 110-180 C for a time range of 5 min to 2 h with 20 min typically being sufficient. The product from the reaction can be isolated and purified employing standard techniques, such as solvent extraction, chromatography, crystallization, distillation and the like.

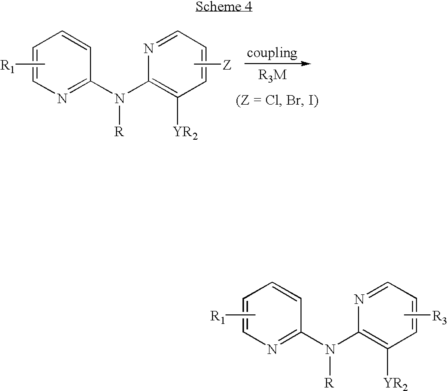

In another embodiment of the present invention, a suitably substituted 2-hydroxypyridine is coupled to an appropriately funtionalized 2-halopyridine (Scheme 5). The reaction may be effected thermally in the temperature range of 160-200 C. Typically the reaction is carried out in the presence of base (e.g. Cs₂CO₃, K₂CO₃, etc. . . .) in a suitable solvent, such as DMF, DMSO, DMAC and the like, and takes from 1 h up to about 72 h with 18 h typically being sufficient (see for example Cherng, Yie-Jia Tetrahedron 2002 58 (24), 4931-4936; Hill, A. J.; McGraw, W. J. J. Org. Chem. 1949, 14, 783-5). The product from the reaction can be isolated and purified employing standard techniques, such as solvent extraction, chromatography, crystallization, distillation and the like.

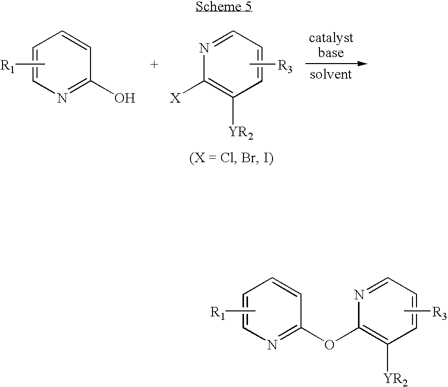

Exemplifying the invention is the use of the compounds disclosed in the Examples and herein. Specific compounds within the present invention include a compound which selected from the group consisting of the compounds disclosed in the following Examples and pharmaceutically acceptable salts thereof and individual diastereomers thereof.

### EXAMPLE 1

### 3-methoxy-N-(6-methylpyridin-2-yl)pyridin-2-amine

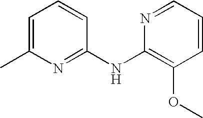

A mixture of 2-bromo-3-hydroxypyridine (1 g, 5.75 mmol), iodomethane (0.7 mL, 11.5 mmol), and K₂CO₃ (1.6 g, 11.5 mmol) in DMF (30 mL) was heated to 40° C. for 2 hr. The solvent was then removed in vacuo and the dark residue was partitioned between EtOAc and brine. The organic layer was washed with brine (2×), dried over Na₂SO₄, filtered, and evaporated to dryness to afford 2-bromo-3-methoxypyridine. ¹H NMR (CDCl₃, 500 MHz) δ 7.98 (d, 1H), 7.20 (t, 1H), 7.14 (d, 1H), 3.94 (s, 3H).

General Procedure A: Microwave Assisted Buchwald Amination:

A mixture of 2-bromo-3-methoxypyridine (200 mg, 1.06 mmol), 6-methylpyridin-2-amine (172 mg, 1.59 mmol), Pd₂(dba)₃ (44 mg, 0.04 mmol), BINAP (52 mg, 0.08 mmol), and sodium tert-butoxide (203 mg, 2.1 mmol) in toluene (4 mL) was placed in a sealed tube and heated in a microwave (Personal Chemistry, Model: Smith Creator) for 10 min at 130° C. The reaction mixture was filtered through a celite pad and rinsed with EtOAc. The residue was purified by flash chromatography on silica gel eluting with a mixture of EtOAc and Hexane to afford 6-methyl-N-pyridin-2-ylpyridin-2-amine. ¹H NMR (CDCl₃, 500 MHz) δ 8.36 (d, 1H), 7.86 (d, 1H), 7.81 (bs, 1H), 7.55 (t, 1H), 6.99 (d, 1H), 6.76 (m, 2H), 3.88 (s, 3H), 2.45 (s, 3H). MS (ESI⁺) 216 (M⁺+1).

### EXAMPLE 2

### 3-(benzyloxy)-N-(6-methylpyridin-2-yl)pyridin-2-amine

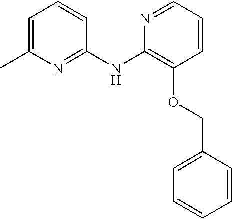

3-(Benzyloxy)-N-(6-methylpyridin-2-yl)pyridin-2-amine was obtained by following procedure A using 6-methylpyridin-2-amine and 3-(Benzyloxy)-2-bromopyridine (synthesized as described for 2-bromo-3-methoxypyridine using benzyl bromide). ¹H NMR (CDCl₃, 500 MHz) δ 8.36 (d, 1H), 7.90 (d, 1H), 7.83 (bs, 1H), 7.54 (t, 1H), 7.38 (m, 5H), 7.01 (d, 1H), 6.70 (m, 2H), 5.14 (s, 2H), 2.43 (s, 3H). MS (ESI) 292 (M+H).

### EXAMPLE 3

### 3-ethoxy-N-(6-methoxypyridin-2-yl)pyridin-2-amine

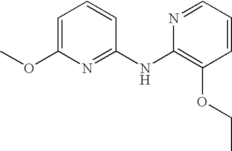

3-ethoxy-N-(6-methoxypyridin-2-yl)pyridin-2-amine was obtained by following procedure A using 6-methoxypyridin-2-amine and 2-bromo-3-ethoxypyridine (synthesized as described for 2-bromo-3-methoxypyridine using ethyl iodide). ¹H NMR (CDCl₃, 500 MHz) δ 8.12 (d, 1H), 7.85 (d, 1H), 7.67 (bs, 1H), 7.56 (t, 1H), 6.94 (d, 1H), 6.72 (m, 1H), 6.28 (d, 1H), 4.07 (q, 2H), 4.07 (s, 3H), 1.46 (t, 3H). MS (ESI) 246 (M+H).

### EXAMPLE 4

### 3-ethoxy-N-(6-methylpyridin-2-yl)pyridin-2-amine

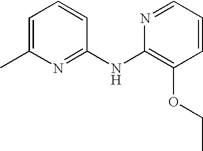

3-Ethoxy-N-(6-methylpyridin-2-yl)pyridin-2-amine was obtained by following procedure A using 6-methylpyridin-2-amine and 2-bromo-3-ethoxypyridine. ¹H NMR (CDCl₃, 500 MHz) δ 8.37 (d, 1H), 7.84 (d, 1H), 7.79 (bs, 1H), 7.55 (t, 1H), 6.96 (d, 1H), 6.72 (m, 2H), 4.09 (q, 2H), 2.46 (s, 3H), 1.48 (t, 3H). MS (ESI) 230 (M+H).

### EXAMPLE 5

### N-(6-methylpyridin-2-yl)-3-propoxypyridin-2-amine

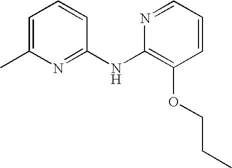

N-(6-Methylpyridin-2-yl)-3-propoxypyridin-2-amine was obtained by following procedure A using 6-methylpyridin-2-amine and 2-bromo-3-propoxypyridine (synthesized as described for 2-bromo-3-methoxypyridine using propyl iodide). ¹H NMR (CDCl₃, 500 MHz) δ 836 (d, 1H), 7.84 (d, 1H), 7.77 (bs, 1H), 7.54 (t, 1H), 6.98 (d, 1H), 6.72 (m, 2H), 3.98 (q, 2H), 2.45 (s, 3H), 1.87 (m, 2H), 1.06 (t, 3H). MS (ESI) 244 (M+H).

### EXAMPLE 6

### N-(6-methylpyridin-2-yl)furo[2,3-c]pyridin-7-amine

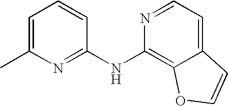

N-(6-Methylpyridin-2-yl)furo[2,3-c]pyridin-7-amine was obtained by following procedure A using 6-methylpyridin-2-amine and 7-bromofuro[2,3-c]pyridine. ¹H NMR (CDCl₃, 500 MHz) δ 8.40 (d, 1H), 8.04 (d, 1H), 7.68 (m, 2H), 7.59 (t, 1H), 7.26 (d, 1H), 6.74 (s, 2H), 2.51 (s, 3H). MS (ESI) 226 (M+H).

### EXAMPLE 7

### 3-ethoxy-N-pyridin-2-ylpyridin-2-amine

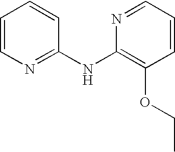

3-Ethoxy-N-pyridin-2-ylpyridin-2-amine was obtained by following procedure A using 2-aminopyridine and 2-bromo-3-ethoxypyridine. ¹H NMR (CDCl₃, 500 MHz) δ 8.58 (d, 1H), 8.26 (d, 1H), 7.89 (bs, 1H), 7.87 (d, 1H), 7.67 (t, 1H), 6.99 (d, 1H), 6.87 (m, 1H), 6.77 (m, 1H), 4.13 (q, 2H), 1.51 (t, 3H). MS (ESI) 216 (M+H).

### EXAMPLE 8

### methyl 6-[(3-ethoxypyridin-2-yl)amino]pyridine-2-carboxylate

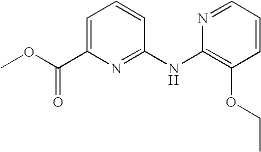

Methyl 6-[(3-ethoxypyridin-2-yl)amino]pyridine-2-carboxylate was obtained by following procedure A using methyl 6-aminopyridine-2-carboxylate and 2-bromo-3-ethoxypyridine. ¹H NMR (CDCl₃, 500 MHz) δ 8.83 (d, 1H), 8.09 (bs, 1H), 7.85 (d, 1H), 7.79 (t, 1H), 7.69 (d, 1H), 7.00 (d, 1H), 6.79 (m, 1H), 4.13 (q, 2H), 3.96 (s, 3H), 1.51 (t, 3H). MS (ESI) 274 (M+H).

### EXAMPLE 9

### 4-methyl-N-(6-methylpyridin-2-yl)-3-(methylthio)pyridin-2-amine

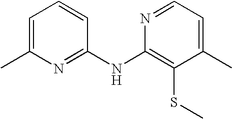

4-Methyl-N-(6-methylpyridin-2-yl)-3-(methylthio)pyridin-2-amine was obtained by following procedure A using 2-bromo-6-methylpyridine and 4-methyl-3-(methylthio) pyridin-2-amine. ¹H NMR (CDCl₃, 500 MHz) δ 8.83 (bs, 1H), 8.29 (d, 1H), 8.09 (d, 1H), 7.56 (t, 1H), 6.76 (d, 1H), 6.70 (d, 1H), 2.53 (s, 3H), 2.47 (s, 3H), 2.12 (s, 3H). MS (ESI) 246 (M+H).

### EXAMPLE 10

### ({2-[(6-methylpyridin-2-yl)amino]pyridin-3-yl}oxy)acetonitrile

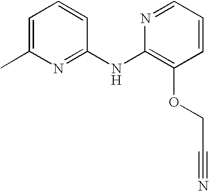

({2-[(6-Methylpyridin-2-yl)amino]pyridin-3-yl}oxy)acetonitrile was obtained by following procedure A using 6-methylpyridin-2-amine and [(2-bromopyridin-3-yl)oxy]acetonitrile (synthesized as described for 2-bromo-3-methoxypyridine using bromoacetonitrile). ¹H NMR (CDCl₃, 500 MHz) δ 8.34 (d, 1H), 8.00 (d, 1H), 7.66 (bs, 1H), 7.56 (t, 1H), 7.16 (d, 1H), 6.82 (m, 1H), 6.77 (d, 1H), 4.78 (s, 2H), 2.47 (s, 3H). MS (ESI) 241 (M+H).

### EXAMPLE 11

### N³-ethyl-N²-(6-methylpyridin-2-yl)pyridine-2,3-diamine

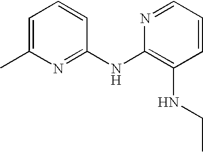

A mixture of 2-bromopyridin-3-amine (1 g, 5.8 mmol), di-tert-butyl dicarbonate (1.76 g, 8 mmol), DMAP (141 mg, 1.16 mmol), and ethyl(diisopropyl)amine (2 mL, 11.6 mmol) in CH₂Cl₂ (40 mL) was stirred at room temperature for 18 hr. The solvent was removed in vacuo and the residue was purified by silica gel chromatography (ethyl acetate/hexanes) to afford tert-butyl (2-bromopyridin-3-yl)carbamate). ¹H NMR (CDCl₃, 500 MHz) δ 8.40 (d, 1H), 8.02 (d, 1H), 7.13 (t, 1H), 7.08 (bs, 1H), 1.54 (s, 9H).

A mixture of tert-butyl(2-bromopyridin-3-yl)carbamate (300 mg, 1.1 mmol), EtI (0.14 mL, 1.65 mmol), and NaH (32 mg, 1.32 mmol) in DMF (10 mL) was heated to 80° C. for 18 hr. The solvent was then removed in vacuo and the residue was partitioned between ethyl acetate and brine. The organic layer was washed with brine (2×), dried (Na₂SO₄) and concentrated. The crude residue was purified by silica gel chromatography (ethyl acetate/hexanes) to afford tert-butyl(2-bromopyridin-3-yl)ethylcarbamate. ¹H NMR (CDCl₃, 500 MHz) δ 8.30 (d, 1H), 7.49 (d, 1H), 7.29 (t, 1H), 3.83 (m, 1H), 3.46 (m, 1H), 1.48 (s, 2H), 1.35 (s, 7H), 1.13 (t, 3H).

N³-Ethyl-N²-(6-methylpyridin-2-yl)pyridine-2,3-diamine was obtained by following procedure A using 6-methylpyridin-2-amine and tert-butyl(2-bromopyridin-3-yl)ethylcarbamate (the Boc group was cleaved in the reaction). ¹H NMR (CDCl₃, 500 MHz) δ 7.78 (d, 1H), 7.48 (m, 2H), 6.91 (m, 2H), 6.79 (bs, 1H), 6.68 (t, 1H), 3.64 (bs, 1H), 3.15 (q, 2H), 2.43 (s, 3H), 1.27 (t, 3H). MS (ESI) 229 (M+H).

### EXAMPLE 12

### 3-ethoxy-4-methyl-N-(6-methylpyridin-2-yl)pyridin-2-amine

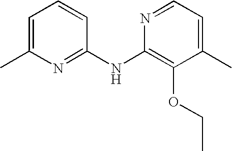

To a solution of 3-hydroxypyridine (9.5 g, 0.1 mol) in aq. NaOH (50 mL, 4M) at −20° C. was added a solution of Br₂ (10.5 mL, 0.2 mol) in aq. NaOH (175 mL, 2.7M). After 30 min, the reaction mixture was acidified with conc. HCl until pH˜5. The precipitate was removed by filtration and the filtrate was adjusted to a pH˜2 with conc HCl. The aqueous layer was extracted with ethyl acetate (5×), dried (MgSO₄) and concentrated to give an orange solid. The solid was washed with Et₂O and the washings were concentrated in vacuo to give a mixture of the desired 2,4 dibromo-3-hydroxypyridine (major) and the 2-6-dibromo-3-hydroxypyridine isomer (minor). The mixture was used without further purification.

To a mixture of the crude dibromides (2 g) and K₂CO₃ (1.6 g, 11.8 mmol) in DMF (30 mL) was added bromoethane (0.88 mL, 11.8 mmol). The reaction mixture was heated to 70° C. for 90 min, cooled, diluted with H₂O, and extracted with ethyl acetate (4×). The combined organic extracts were dried (MgSO₄) and concentrated. The crude residue was purified by silica gel chromatography (1:1 Et₂O/hexanes) to give 2,4-dibromo-3-ethoxypyridine as a yellow oil judged to be ˜75% pure by ¹H NMR analysis.

The bipyridyl amine was obtained by following general procedure A using 6-methylpyridin-2-amine and 2,4-dibromo-3-ethoxypyridine. ¹H NMR (CDCl₃, 500 MHz) δ 8.20 (br s, 1H), 7.84 (d, 1H), 7.61 (t, 1H), 7.02 (d, 1H), 6.80 (d, 1H), 3.19 (q, 2H), 2.03 (s, 3H), 1.52 (t, 1H). MS (ESI) 308 (M⁺).

A mixture of this bromide (50 mg, 0.16 mmol), methyl boronic acid (29 mg, 0.48 mmol), Pd(PPh₃)₄ (19 mg, 0.016 mmol), and CsF (73 mg, 0.48 mmol) in DME (2 mL) was placed in a sealed tube and reacted as described in Procedure B. The crude mixture was purified by flash chromatography on silica gel (ethyl acetate/hexanes) to afford 3-ethoxy-4-methyl-N-(6-methylpyridin-2-yl)pyridin-2-amine. ¹H NMR (CDCl₃, 500 MHz) δ 8.11 (bs 1H), 7.68 (d, 1H), 7.36 (t, 1H), 6.52 (d, 1H), 6.45 (bs, 1H), 3.78 (q, 2H), 2.27 (s, 3H), 2.07 (s, 3H), 1.28 (t, 3H). MS (ESI) 244 (M+H).

### EXAMPLE 13

### 3-ethoxy-2-[(6-methylpyridin-2-yl)amino]isonicotinonitrile

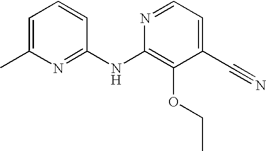

A mixture 4-bromo-3-ethoxy-N-(6-methylpyridin-2-yl)pyridin-2-amine (50 mg, 0.16 mmol), NaCN (16 mg, 0.32 mmol), Pd(PPh₃)₄ (10 mg, 0.008 mmol), and CuI (3 mg, 0.016 mmol) in CH₃CN was heated in a microwave (Personal Chemistry, Model: Smith Creator) for 60 min at 180° C. The reaction mixture was filtered through celite and concentrated. The crude residue was purified by flash chromatography on silica gel (ethyl acetate/hexanes) to afford 3-ethoxy-2-[(6-methylpyridin-2-yl)amino]isonicotinonitrile. ¹H NMR (CDCl₃, 500 MHz) δ 8.28 (bs, 1H), 7.99 (bs, 2H), 7.61 (t, 1H), 6.82 (m, 2H), 4.50 (q, 2H), 2.49 (s, 3H), 1.54 (t, 3H). MS (ESI) 255 (M+H).

### EXAMPLE 14

### 3-ethoxy-N-[6-(trifluoromethyl)pyridin-2-yl]pyridin-2-amine

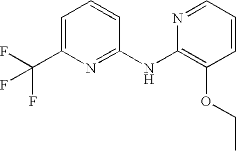

3-Ethoxypyridin-2-amine was obtained by hydrogenation of 3-ethoxy-2-nitropyridine (synthesized as described for 2-bromo-3-methoxypyridine using 2-nitropyridin-3-ol and ethyl iodide) in the presence of Pd/C. ¹H NMR (CDCl₃, 500 MHz) δ 7.64 (d, 1H), 6.89 (d, 1H), 6.59 (t, 1H), 4.64 (bs, 2H), 4.05 (q, 2H), 1.44 (t, 3H).

3-Ethoxy-N-[6-(trifluoromethyl)pyridin-2-yl]pyridin-2-amine was obtained by following procedure A using 3-ethoxypyridin-2-amine and 2-chloro-6-(trifluoromethyl)pyridine. ¹H NMR (CDCl₃, 500 MHz) δ 8.85 (d, 1H), 8.08 (bs, 1H), 7.91 (d, 1H), 7.84 (t, 1H), 7.28 (d, 1H), 7.11 (d, 1H), 6.87 (m, 1H), 4.16 (q, 2H), 1.55 (t, 3H). MS (ESI) 284 (M+H).

### EXAMPLE 15

### 5-bromo-3-ethoxy-N-(6-methylpyridin-2-yl)pyridin-2-amine

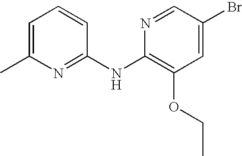

To a solution of 3-ethoxypyridin-2-amine (1.5 g, 11 mmol) in CH₃CN (40 mL) and CH₂Cl₂ (100 mL) was added B₂ (0.7 mL, 13 mmol). The resulting dark mixture was stirred at room temperature for 1.25 hr and solid NaHCO₃ (4.5 g, 54 mmol) was added. The mixture was stirred an additional 1 hr at room temperature followed by addition of an aqueous sodium bisulfite solution. The layers were separated and the aqueous layer was extracted with CH₂Cl₂ (3×). The combined organics were dried (Na₂SO₄) and concentrated to afford 5-bromo-3-ethoxypyridin-2-amine. MS (ESI) 218 (M+H).

A mixture of 5-bromo-3-ethoxypyridin-2-amine (2 g, 9.2 mmol), 2-bromo-6-methylpyridine (1.32 g, 7.7 mmol), Pd₂(dba)₃ (211 mg, 0.23 mmol), BINAP (287 mg, 0.46 mmol), and sodium tert-butoxide (1.47 g, 15.4 mmol) in toluene (50 mL) was placed in a sealed tube and heated to 110° C. for 18 hr. The reaction mixture was filtered through celite and concentrated. The residue was purified by flash chromatography on silica gel (ethyl acetate/hexanes) to afford 5-bromo-3-ethoxy-N-(6-methylpyridin-2-yl)pyridin-2-amine. ¹H NMR (CDCl₃, 500 MHz) δ 8.09 (d, 1H), 7.70 (s, 1H), 7.58 (bs, 1H), 7.40 (t, 1H), 6.89 (s, 1H), 6.58 (d, 1H), 3.91 (q, 2H), 2.28 (s, 3H), 1.32 (t, 3H). MS (ESI) 309 (M+H).

### EXAMPLE 16

### 5-ethoxy-N-(6-methylpyridin-2-yl)-3,4′-bipyridin-6-amine

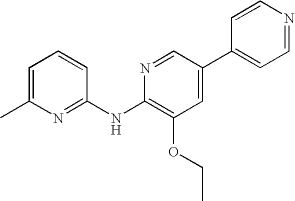 General Procedure B: Microwave Assisted Suzuki Coupling:

A mixture of 5-bromo-3-ethoxy-N-(6-methylpyridin-2-yl)pyridin-2-amine (50 mg, 0.16 mmol), 4-pyridyl boronic acid (30 mg, 0.24 mmol), PdCl₂(PPh₃)₂ (11 mg, 0.016 mmol), and potassium carbonate (44 mg, 0.32 mmol) in DME/H₂O (5:1, 2 mL) was placed in a sealed tube and heated in a microwave (Personal Chemistry, Model: Smith Creator) for 15 min at 160° C. The reaction mixture was diluted with EtOAc and washed with H₂O (2×). The organic phase was dried over Na₂SO₄, filtered, and evaporated to dryness. The residue was purified by flash chromatography on silica gel eluting with a mixture of EtOAc and Hexane to afford 5-Ethoxy-N-(6-methylpyridin-2-yl)-3,4′-bipyridin-6-amine. ¹H NMR (CDCl₃, 500 MHz) δ 8.58 (d, 2H), 8.42 (bs, 1H), 8.11 (d, 1H), 7.59 (t, 1H), 7.41 (d, 2H), 7.17 (s, 1H), 7.17 (d, 1H), 4.15 (q, 2H), 2.48 (s, 3H), 1.52 (t, 3H). MS (ESI⁺) 307 (M⁺+1).

### EXAMPLE 17

### 5-ethoxy-N-(6-methylpyridin-2-yl)-3,3′-bipyridin-6-amine

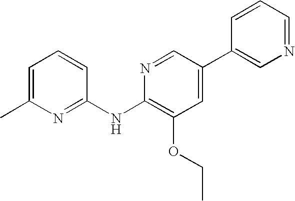

(5-Ethoxy-N-(6-methylpyridin-2-yl)-3,3′-bipyridin-6-amine was obtained by following procedure B using 5-bromo-3-ethoxy-N-(6-methylpyridin-2-yl)pyridin-2-amine and 3-pyridyl boronic acid. ¹H NMR (CDCl₃, 500 MHz) δ 8.74 (s, 1H), 8.52 (d, 1H). 8.37 (bs, 1H), 8.01 (s, 1H), 7.76 (dt, 1H), 7.60 (m, 1H), 7.40 (td, 1H), 7.29 (td, 1H), 7.12 (s, 1H), 6.70 (d, 1H), 4.13 (q, 2H), 2.43 (s, 3H), 1.50 (t, 3H). MS (ESI) 307 (M+H).

### EXAMPLE 18

### 3-ethoxy-5-methyl-N-(6-methylpyridin-2-yl)pyridin-2-amine

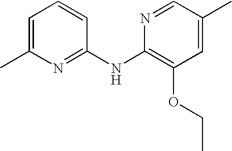

A mixture of 5-bromo-3-ethoxy-N-(6-methylpyridin-2-yl)pyridin-2-amine (50 mg, 0.16 mmol), methyl boronic acid (29 mg, 0.48 mmol), Pd(PPh₃)₄ (19 mg, 0.016 mmol), and CsF (73 mg, 0.48 mmol) in DME (2 mL) was placed in a sealed tube and reacted as described in procedure B. The crude mixture was purified by reverse phase preparative HPLC to obtain 11 mg of 3-ethoxy-5-methyl-N-(6-methylpyridin-2-yl)pyridin-2-amine. ¹H NMR (MeOD, 500 MHz) δ 7.97(t, 1H), 7.82 (s, 1H), 7.52 (s, 1H), 7.46 (d, 1H), 7.11 (d, 1H), 4.33 (q, 2H), 2.68 (s, 3H), 2.39 (s, 3H), 1.54 (t, 3H).

### EXAMPLE 19

### 3-ethoxy-N²-(6-methylpyridin-2-yl)pyridine-2,5-diamine

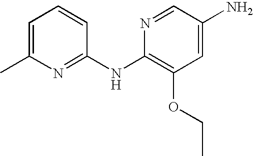

N⁵-(Diphenylmethylene)-3-ethoxy-N²-(6-methylpyridin-2-yl)pyridine-2,5-diamine was obtained by following procedure A using 5-bromo-3-ethoxy-N-(6-methylpyridin-2-yl)pyridin-2-amine and 1,1-diphenylmethanimine.

A mixture of N⁵-(Diphenylmethylene)-3-ethoxy-N²-(6-methylpyridin-2-yl)pyridine-2,5-diamine and hydroxylamine hydrochloride in MeOH was stirred at room temperature for 18 hr. The solvent was removed in vacuo and the residue was partitioned between Ethyl acetate and a 1M aqueous HCl solution. The organic layer was extracted with additional 1M HCl (2×). The combined acidic extracts were neutralized with 5M aqueous NaOH, extracted with Ethyl acetate (3×), dried (MgSO₄) and concentrated to afford 3-ethoxy-N²-(6-methylpyridin-2-yl)pyridine-2,5-diamine. ¹H NMR (CDCl₃, 500 MHz) δ 8.25 (d, 1H), 7.54 (t, 1H), 7.42 (s, 1H), 6.67 *d, 1H), 6.55 (s, 1H), 4.10 (q, 2H), 2.47 (s, 3H), 1.49 (t, 3H). MS (ESI) 307 (M+H).

### EXAMPLE 20

### 5-ethoxy-6-[(6-methylpyridin-2-yl)amino]nicotinonitrile

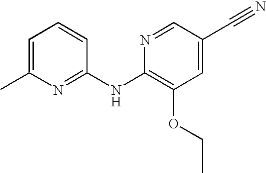

A mixture 5-bromo-3-ethoxy-N-(6-methylpyridin-2-yl)pyridin-2-amine (100 mg, 0.32 mmol), KCN (42 mg, 0.64 mmol), Pd(PPh₃)₄ (19 mg, 0.016 mmol), CuI (6 mg, 0.032 mmol) in CH₃CN was heated in a microwave (Personal Chemistry, Model: Smith Creator) for 1 h at 180° C. The reaction mixture was filtered through celite and concentrated. The crude residue was purified by silica gel chromatography with a mixture of Ethyl acetate and hexanes to give 5-ethoxy-6-[(6-methylpyridin-2- yl)amino]nicotinonitrile as a white solid. ¹H NMR (CDCl₃, 500 MHz) δ 8.36 (bd, 1H), 8.19 (d, 1H), 8.07 (bs, 1H), 7.64 (t, 1H), 7.09 (s, 1H), 6.86 (d, 1H), 4.15 (q, 2H), 2.57 (s, 3H), 1.64 (t, 3H). MS (ESI) 255 (M+H).

### EXAMPLE 21

### 5′-ethoxy-N-(6-methylpyridin-2-yl)-2,3′-bipyridin-6′-amine

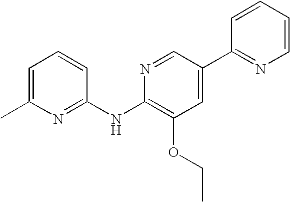

A mixture of 5-bromo-3-ethoxy-N-(6-methylpyridin-2-yl)pyridin-2-amine (100 mg, 0.32 mmol), 2-tributylstannylpyridine (177 mg, 0.48 mmol), and Pd(PPh₃)₄ (37 mg, 0.032 mmol) in DMF (10 mL) was heated to 120° C. for 15 hr. The resulting black mixture was filtered through celite and concentrated. The crude was purified by silica gel chromatography (ethyl acetate/hexanes) to afford 5′-ethoxy-N-(6-methylpyridin-2-yl)-2,3′-bipyridin-6′-amine as a yellow oil. ¹H NMR (CDCl₃, 500 MHz) δ 8.66 (d, 1H), 8.45 (d, 1H), 8.42 (s, 1H), 8.00 (bs, 1H), 7.82 (s, 1H), 7.73 (m, 2H), 7.60 (t, 1H), 7.20 (m, 1H), 6.79 (d, 1H), 4.27 (q, 2H), 2.49 (s, 3H), 1.53 (t, 3H). MS (ESI) 307 (M+H).

### EXAMPLE 22

### 5-ethoxy-6′-methoxy-N-(6-methylpyridin-2-yl)-3,3′-bipyridin-6-amine

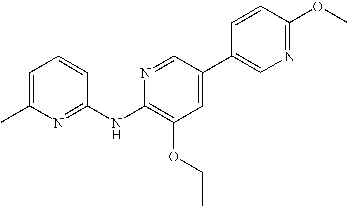

5-Ethoxy-6′-methoxy-N-(6-methylpyridin-2-yl)-3,3′-bipyridin-6-amine was obtained by following procedure B using 3-(4-methoxy)-pyridyl boronic acid,. ¹H NMR (CDCl₃, 500 MHz) δ 8.42 (bm, 1H), 8.36 (s, 1H), 8.04 (s, 1H), 7.76 (dd, 1H), 7.61 (bs, 1H), 7.15 (s, 1H), 6.85 (d, 1H), 6.79 (d, 1H), 4.22 (q, 2H), 4.00 (s, 1H), 2.52 (s, 3H), 1.56 (t, 3H). MS (ESI) 337 (M+H).

### EXAMPLE 23

### 3-{-5-ethoxy-6-[(6-methylpyridin-2-yl)amino]pyridin-3-yl}benzonitrile

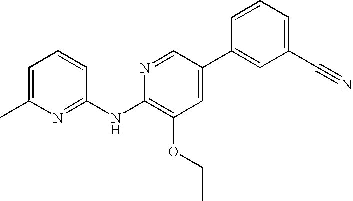

3-{5-Ethoxy-6-[(6-methylpyridin-2-yl)amino]pyridin-3-yl}benzonitrile was obtained as a white solid by following procedure B using 3-cyanophenyl boronic acid. ¹H NMR (CDCl₃, 500 MHz) δ 8.38 (d, 1H), 8.07 (d, 1H), 7.89 (s, 1H), 7.80 (s, 1H), 7.66 (d, 1H), 7.61 (m, 2H), 7.52 (t, 1H), 7.14 (d, 1H), 6.78 (d, 1H), 4.19 (q, 2H), 2.48 (s, 3H), 1.56 (t, 3H). MS (ESI) 331 (M+H).

### EXAMPLE 24

### 3-ethoxy-5-(3-fluorophenyl)-N-(6-methylpyridin-2-yl)pyridin-2-amine

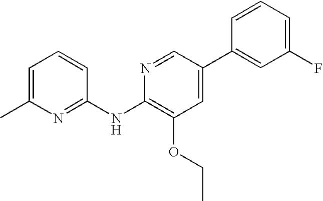

3-Ethoxy-5-(3-fluorophenyl)-N-(6-methylpyridin-2-yl)pyridin-2-amine was obtained as a white solid following procedure B using 3-fluorophenyl boronic acid,. ¹H NMR (CDCl₃, 500 MHz) δ 8.38 (d, 1H), 8.08 (d, 1H), 7.84 (bs, 1H), 7.58 (t, 1H), 7.38 (m, 1H), 7.32 (dt, 1H), 7.22 (dt, 1H), 7.18 (d, 1H), 7.02 (td, 1H), 6.75 (d, 1H), 4.19 (q, 2H), 2.47 (s, 3H), 1.51 (t, 3H). MS (ESI) 324 (M+H).

### EXAMPLE 25

### 3-ethoxy-N-(6-methylpyridin-2-yl)-5-(trimethylstannyl)pyridin-2-amine

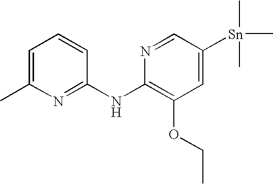 General Procedure C: Thermal Stille Coupling:

A mixture of 5-bromo-3-ethoxy-N-(6-methylpyridin-2-yl)pyridin-2-amine (300 mg, 0.97 mmol), Me₆Sn₂ (638 mg, 1.95 mmol), and Pd(PPh₃)₄ (112 mg, 0.097 mmol) in THF (5 mL) was heated in a sealed tube at 60° C. for 10 hr. The reaction mixture was cooled, diluted with ethyl acetate and poured into a saturated aqueous NaHCO₃. The layers were separated and the organic layer was washed with saturated aqueous NaHCO₃ solution (2×). The organic layer was dried (Na₂SO₄) and concentrated. The crude residue was purified by silica gel chromatography (ethyl acetate/hexanes) to yield 297 mg of 3-ethoxy-N-(6-methylpyridin-2-yl)-5-(trimethylstannyl)pyridin-2-amine as a yellow solid. MS (ESI) 392 (M⁺).

### EXAMPLE 26

### 5′-ethoxy-6′-[(6-methylpyridin-2-yl)amino]-3,3′-bipyridine-5-carbonitrile

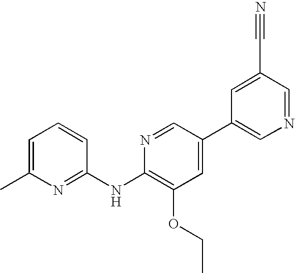

5′-Ethoxy-6′-[(6-methylpyridin-2-yl)amino]-3,3′-bipyridine-5-carbonitrile was obtained following procedure C using 3-ethoxy-N-(6-methylpyridin-2-yl)-5-(trimethylstannyl) pyridin-2-amine and 5-bromonicotinonitrile. ¹H NMR (CDCl₃, 500 MHz) δ 8.85 (s, 1H), 8.68 (s, 1H), 8.27 (bs, 1H), 7.94 (m, 2H), 7.81 (bs, 1H), 7.47 (bt, 1H), 6.99 (s, 1H), 6.66 (d, 1H), 4.09 (q, 2H), 2.35 (s, 3H), 1.43 (t, 3H). MS (ESI) 332 (M+H).

### EXAMPLE 27

### 5-ethoxy-2′-methyl-N-(6-methylpyridin-2-yl)-3,3′-bipyridin-6-amine

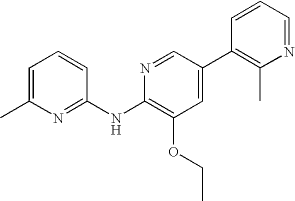

5-Ethoxy-2′-methyl-N-(6-methylpyridin-2-yl)-3,3′-bipyridin-6-amine was obtained by following procedure C using 3-ethoxy-N-(6-methylpyridin-2-yl)-5-(trimethylstannyl) pyridin-2-amine and 3-bromo-2-methylpyridine. ¹H NMR (MeOD, 500 MHz) δ 8.82 (bs, 1H), 8.55 (d, 1H), 8.15 (m, 2H), 8.03 (t, 1H), 7.23 (m, 2H), 7.24 (d, 1H), 4.37 (q, 2H), 2.80 (s, 3H), 2.76 (s, 3H), 1.56 (t, 3H). MS (ESI) 321 (M+H).

### EXAMPLE 28

### 5-ethoxy-6′-methyl-N-(6-methylpyridin-2-yl)-3,3′-bipyridin-6-amine

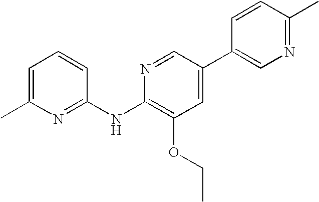 General Procedure D: Microwave Assisted Stille Coupling:

A mixture of 3-ethoxy-N-(6-methylpyridin-2-yl)-5-(trimethyl stannyl) pyridin-2-amine (200 mg, 0.51 mmol), 5-bromo-2-methylpyridine (132 mg, 0.76 mmol), and Pd(PPh₃)₄ (12 mg, 0.01 mmol) in DMF (5 mL) was placed in a sealed tube and heated in a microwave (Personal Chemistry, Model: Smith Creator) for 15 nm at 140° C. The resulting yellow solution was diluted with Ethyl acetate and washed with H₂O (2×). The organic layer was dried (Na₂SO₄) and concentrated. The crude residue was purified by silica gel chromatography (Ethyl acetate/hexane). This product was dissolved in CH₂Cl₂/Et₂O (2 mL, 1:2) and treated with HCl (1M in Et₂O) to obtain the HCl salt of 5-ethoxy-6′-methyl-N-(6-methylpyridin-2-yl)-3,3′-bipyridin-6-amine. ¹H NMR (MeOD, 500 MHz) δ 9.20 (s, 1H), 8.91 (d, 1H), 8.52 (s, 1H), 8.20 (t, 1H), 8.08 (d, 1H), 8.02 (s, 1H), 7.75 (d, 1H), 7.24 (d, 1H), 4.47 (q, 2H), 2.88 (s, 3H), 2.78 (s, 3H), 1.59 (t, 3H). MS (ESI) 321 (M+H).

### EXAMPLE 29

### 5-ethoxy-5′-methyl-N-(6-methylpyridin-2-yl)-3,3′-bipyridin-6-amine

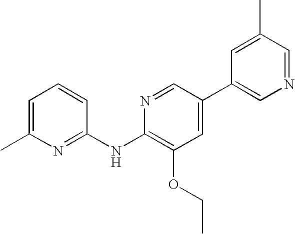

5-Ethoxy-5′-methyl-N-(6-methylpyridin-2-yl)-3,3′-bipyridin-6-amine was obtained by following procedure D using 3-ethoxy-N-(6-methylpyridin-2-yl)-5-(trimethyl stannyl) pyridin-2-amine and 3-bromo-5-methylpyridine. ¹H NMR (MeOD, 500 MHz) δ 9.18 (s, 1H), 8.93 (s, 1H), 8.78 (s, 1H), 8.53 (s, 1H), 8.18 (t, 1H), 8.03 (s, 1H), 7.73 (d, 1H), 7.24 (d, 1H), 4.48 (q, 2H), 2.78 (s, 3H), 2.69 (s, 3H), 1.60 (t, 3H). MS (ESI) 321 (M+H).

### EXAMPLE 30

### 5-ethoxy-4′-methyl-N-(6-methylpyridin-2-yl)-3,3′-bipyridin-6-amine

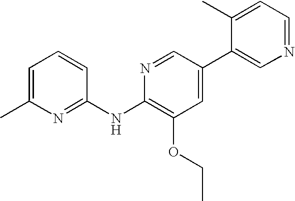

5-Ethoxy-4′-methyl-N-(6-methylpyridin-2-yl)-3,3′-bipyridin-6-amine was obtained by following procedure D using 3-ethoxy-N-(6-methylpyridin-2-yl)-5-(trimethyl stannyl) pyridin-2-amine and 3-bromo-4-methylpyridine. ¹H NMR (MeOD, 500 MHz) δ 8.88 (s, 1H), 8.79 (d, 1H), 8.14 (m, 3H), 7.72 (m, 2H), 7.24 (d, 1H), 4.41 (q, 2H), 2.77 (s, 3H), 2.69 (s, 3H), 1.57 (t, 3H). MS (ESI) 321 (M+H).

### EXAMPLE 31

### 5-ethoxy-2′,6′-dimethyl-N-(6-methylpyridin-2-yl)-3,3′-bipyridin-6-amine

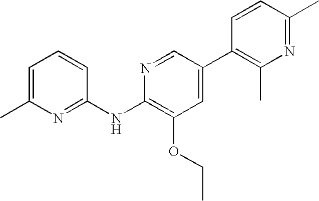

5-Ethoxy-2′,6′-dimethyl-N-(6-methylpyridin-2-yl)-3,3′-bipyridin-6-amine was obtained by following procedure D using 3-ethoxy-N-(6-methylpyridin-2-yl)-5-(trimethyl stannyl) pyridin-2-amine and 3-bromo-2,6-dimethylpyridine. ¹H NMR (MeOD, 500 MHz) δ 8.42 (d, 1H), 8.17 (m, 2H), 7.86 (d, 1H), 7.68 (m, 2H), 7.25 (d, 1H), 4.41 (q, 2H), 2.86 (s, 3H), 2.79 (s, 3H), 2.77 (s, 3H), 1.56 (t, 3H). MS (ESI) 335 (M+H).

### EXAMPLE 32

### 3-ethoxy-N-(6-methylpyridin-2-yl)-5-quinolin-3-ylpyridin-2-amine

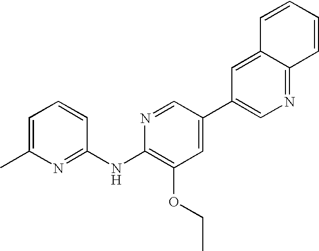

3-Ethoxy-N-(6-methylpyridin-2-yl)-5-quinolin-3-ylpyridin-2-amine was obtained by following procedure D using 3-ethoxy-N-(6-methylpyridin-2-yl)-5-(trimethyl stannyl) pyridin-2-amine and 3-bromoquinoline. ¹H NMR (MeOD, 500 MHz) δ 9.77 (s, 1H), 9.66 (s, 1H), 8.66 (s, 1H), 8.48 (d, 1H), 8.34 (d, 1H), 8.21 (m, 3H), 8.05 (t, 1H), 7.74 (d, 1H), 7.27 (d, 1H), 4.53 (q, 2H), 2.80 (s, 3H), 1.63 (t, 3H). MS (ESI) 357 (M+H).

### EXAMPLE 33

### 3-ethoxy-5-isoquinolin-4-yl-N-(6methylpyridin-2-yl)pyridin-2-amine

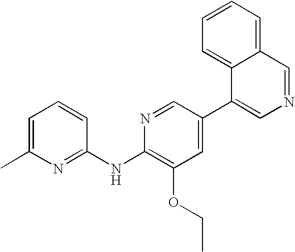

3-Ethoxy-5-isoquinolin-4-yl-N-(6-methylpyridin-2-yl)pyridin-2-amine was obtained by following procedure D using 3-ethoxy-N-(6-methylpyridin-2-yl)-5-(trimethyl stannyl) pyridin-2-amine and 4-bromoisoquinoline. ¹H NMR (MeOD, 500 MHz) δ 9.90 (s, 1H), 8.74 (s, 1H), 8.68 (d, 1H), 8.31 (m, 3H), 8.20 (t, 1H), 8.15 (bt, 1H), 7.87 (s, 1H), 7.76 (d, 1H), 7.26 (d, 1H), 4.42 (q, 2H), 2.78 (s, 3H), 1.57 (t, 3H). MS (ESI) 357 (M+H).

### EXAMPLE 34

### ethyl 5 ′-ethoxy-6′-[(6-methylpyridin-2-yl)amino]-3,3′-bipyridine-5-carboxylate

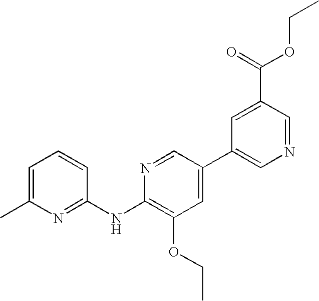

Ethyl 5′-ethoxy-6′-[(6-methylpyridin-2-yl)amino]-3,3′-bipyridine-5-carboxylate was obtained by following procedure D using 3-ethoxy-N-(6-methylpyridin-2-yl)-5-(trimethyl stannyl) pyridin-2-amine and ethyl 5-bromonicotinate. ¹H NMR (MeOD, 500 MHz) δ 9.54 (s, 1H), 9.37 (2s, 2H), 8.57 (s, 1H), 8.20 (t, 1H), 8.14 (s, 1H), 7.75 (d, 1H), 7.25 (d, 1H), 4.58 (q, 2H), 4.50 (q, 2H), 2.79 (s, 3H), 1.61 (t, 3H), 1.47 (t, 3H). MS (ESI) 379 (M+H).

### EXAMPLE 35

### 5,5′-diethoxy-N-(6-methylpyridin-2-yl)-3,3′-bipyridin-6-amine

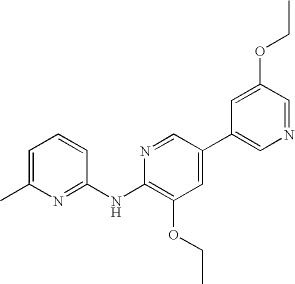

5,5′-Diethoxy-N-(6-methylpyridin-2-yl)-3,3′-bipyridin-6-amine was obtained by following procedure D using 3-ethoxy-N-(6-methylpyridin-2-yl)-5-(trimethyl stannyl) pyridin-2-amine and 3-bromo-5-ethoxypyridine. ¹H N (MeOD, 500 MHz) δ 8.93 (s, 1H), 8.64 (s, 1H), 8.56 (s, 1H), 8.53 (s, 1H), 8.19 (t, 1H), 8.02 (s, 1H), 7.73 (d, 1H), 7.26 (d, 1H), 4.47 (q, 2H), 4.42 (q, 2H), 2.78 (s, 3H), 1.6 (t, 3H), 1.52 (t, 3H). MS (ESI) 351 (M+H).

### EXAMPLE 36

### 5-ethoxy-6′-fluoro-N-(6-methylpyridin-2-yl)-3,3′-bipyridin-6-amine

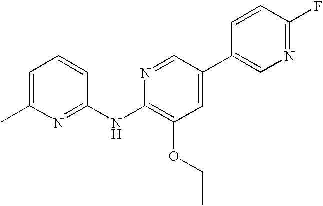

5-Ethoxy-6′-fluoro-N-(6-methylpyridin-2-yl)-3,3′-bipyridin-6-amine was obtained by following procedure D using 3-ethoxy-N-(6-methylpyridin-2-yl)-5-(trimethyl stannyl) pyridin-2-amine and 2-bromo-5-fluoropyridine. ¹H NMR (CDCl₃, 500 MHz) δ 8.56 (s, 1H), 8.33 (s, 1H), 8.30 (t, 1H) 8.12 (t, 1H), 7.85 (s, 1H), 7.63 (d, 1H), 7.22 (m, 2H), 4.42 (q, 2H), 2.75 (s, 3H), 1.58 (t, 3H). MS (ESI) 325 (M+H).

### EXAMPLE 37

### 5′-bromo-5-ethoxy-N-(6-methylpyridin-2-yl)-3,3′-bipyridin-6-amine

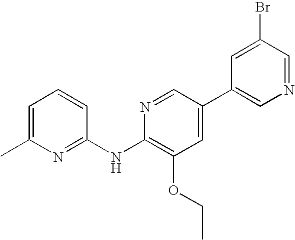

5′-Bromo-5-ethoxy-N-(6-methylpyridin-2-yl)-3,3′-bipyridin-6-amine was obtained by following procedure D using 3-ethoxy-N-(6-methylpyridin-2-yl)-5(trimethyl stannyl) pyridin-2-amine and 3,5-dibromo pyridine. ¹H NMR (MeOD, 500 MHz) δ 8.72 (s, 1H), 8.62 (s, 1H), 8.39 (d, 1H), 8.06 (s, 1H), 7.95 (s, 1H), 7.90 (bs, 1H), 7.55 (t, 1H), 7.09 (s, 1H), 6.79 (d, 1H), 4.19 (q, 2H), 2.48 (s, 3H), 1.53 (t, 3H). MS (ESI) 386 (M+H).

### EXAMPLE 38

### 5′-ethoxy-6′-[(6-methylpyridin-2-yl)amino]-3,3′-bipyridine-2-carbonitrile

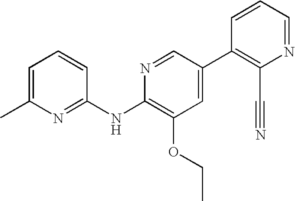

5′-Ethoxy-6′-[(6-methylpyridin-2-yl)amino]-3,3′-bipyridine-2-carbonitrile was obtained by following procedure D using 3-ethoxy-N-(6-methylpyridin-2-yl)-5-(trimethyl stannyl) pyridin-2-amine and 3-bromopyridine-2-carbonitrile. ¹H NMR (MeOD, 500 MHz) δ 8.75 (s, 1H), 8.29 (s, 1H), 8.17 (m, 2H), 7.89 (s, 1H), 7.81 (m, 1H), 7.76 (d, 1H), 7.23 (d, 1H), 4.43 (q, 2H), 2.77 (s, 3H), 1.60 (t, 3H). MS (ESI) 332 (M+H).

### EXAMPLE 39

### 5-ethoxy-5′-methoxy-N-(6-methylpyridin-2-yl)-3,3′-bipyridin-6-amine

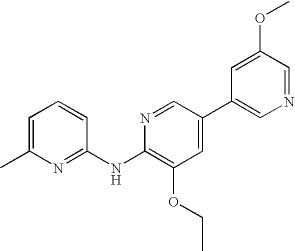

5-Ethoxy-5′-methoxy-N-(6-methylpyridin-2-yl)-3,3′-bipyridin-6-amine was obtained by following procedure D using 3-ethoxy-N-(6-methylpyridin-2-yl)-5(trimethyl stannyl) pyridin-2-amine and 3-bromo-5-methoxypyridine. ¹H NMR (MeOD, 500 MHz) δ 8.94 (s, 1H), 8.67 (s, 1H), 8.59 (s, 1H), 8.53 (s, 1H), 8.19 (t, 1H), 8.03 (s, 1H), 7.73 (d, 1H), 7.23 (d, 1H), 4.46 (q, 2H), 4.17 (s, 3H), 2.78 (s, 3H), 1.60 (t, 3H). MS (ESI) 337 (M+H).

### EXAMPLE 40

### 5-ethoxy-N-(6-methylpyridin-2-yl)-5′-phenyl-3,3′-bipyridin-6-amine

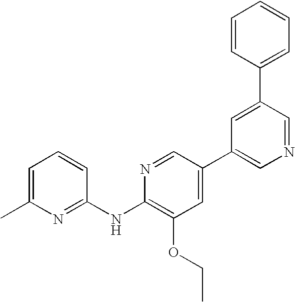

5-Ethoxy-N-(6-methylpyridin-2-yl)-5′-phenyl-3,3′-bipyridin-6-amine was obtained by following procedure D using 3-ethoxy-N-(6-methylpyridin-2-yl)-5-(trimethyl stannyl) pyridin-2-amine and 3-bromo-5-phenylpyridine. ¹H NMR (MeOD, 500 MHz) δ 9.31 (s, 1H), 9.25 (s, 1H), 9.22 (s, 1H), 8.62 (s, 1H), 8.19 (t, 1H), 8.13 (s, 1H), 7.97 (d, 2H), 7.74 (d, 1H), 7.64 (m, 3H), 7.27 (d, 1H), 4.51 (q, 2H), 2.79 (s, 3H), 1.60 (t, 3H). MS (ESI) 383 (M+H).

### EXAMPLE 41

### 3-ethoxy-N-(6-methylpyridin-2-yl)-5-pyrazin-2-ylpyridin-2-amine

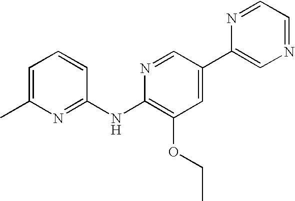

3-Ethoxy-N-(6-methylpyridin-2-yl)-5-pyrazin-2-ylpyridin-2-amine was obtained by following procedure D using 3-ethoxy-N-(6-methylpyridin-2-yl)-5-(trimethyl stannyl) pyridin-2-amine and 2-iodopyrazine. ¹H NMR (MeOD, 500 MHz) δ 9.35 (s, 1H), 8.90 (s, 1H), 8.83 (s, 1H), 8.67 (s, 1H), 8.26 (s, 1H), 8.16 (t, 1H), 7.70 (d, 1H), 7.25 (d, 1H), 4.46 (q, 2H), 2.78 (s, 3H), 1.60 (t, 3H). MS (ESI) 308 (M+H).

### EXAMPLE 42

### 5-ethoxy-N-(6-methylpyridin-2-yl)-5′-(phenylsulfonyl)-3,3′-bipyridin-6-amine

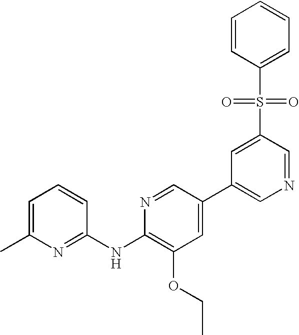

5-Ethoxy-N-(6-methylpyridin-2-yl)-5′-(phenylsulfonyl)-3,3′-bipyridin-6-amine was obtained by following procedure D using 3-ethoxy-N-(6-methylpyridin-2-yl)-5-(trimethyl stannyl) pyridin-2-amine and 3-bromo-5-(phenylsulfonyl)pyridine. ¹H NMR (MeOD, 500 MHz) δ 9.34 (s, 1H), 9.30 (s, 1H), 9.00 (s, 1H), 8.45 (s, 1H), 8.16 (m, 3H), 7.97 (s, 1H), 7.73 (m, 2H), 7.65 (t, 2H), 7.25 (d, 1H), 4.45 (q, 2H), 2.77 (s, 3H), 1.59 (t, 3H). MS (ESI) 447 (M+H).

### EXAMPLE 43

### 5 ethoxy-6′-[(6-methylpyridin-2-yl)amino]-3,3′-bipyridine-5-sulfonamide

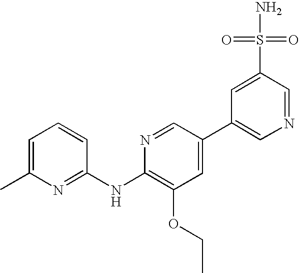

5′-Ethoxy-6′-[(6-methylpyridin-2-yl)amino]-3,3′-bipyridine-5-sulfonamide was obtained by following procedure D using 3-ethoxy-N-(6-methylpyridin-2-yl)-5-(trimethyl stannyl) pyridin-2-amine and 5-bromopyridine-3-sulfonamide. ¹H NMR (DMSO-d6, 500 MHz) δ 10.73 (bs, 1H), 9.23 (s, 1H), 8.99 (s, 1H), 8.62 (s, 1H), 8.46 (s, 1H), 8.16 (m, 2H), 8.04 (s, 1H), 7.75 (bs, 2H), 7.23 (d, 1H), 4.42 (q, 2H), 2.66 (s, 3H), 1.48 (t, 3H). MS (ESI) 386 (M+H).

### EXAMPLE 44

### 5-ethoxy-N⁶′, N⁶′-dimethyl-N⁶-(6-methylpyridin-2-yl)-3,3′-bipyridine-6,6′-diamine

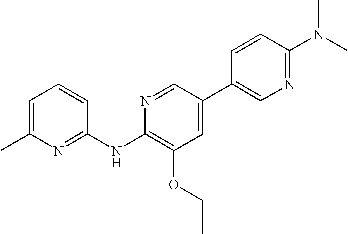

5-Ethoxy-N⁶′,N⁶′-dimethyl-N⁶-(6-methylpyridin-2-yl)-3,3′-bipyridine-6,6′-diamine was obtained by following procedure D using 3-ethoxy-N-(6-methylpyridin-2-yl)-5-(trimethyl stannyl) pyridin-2-amine and 5-bromo-N,N-dimethylpyridin-2-amine. ¹H NMR (MeOD, 500 MHz) δ 8.40 (dd, 1H), 8.35 (s, 1H), 8.29 (s, 1H), 8.14 (t, 1H), 7.85 (s, 1H), 7.66 (d, 1H), 7.41 (d, 1H), 7.21 (d, 1H), 4.44 (q, 2H), 3.36 (s, 6H), 2.75 (s, 3H), 1.58 (t, 3H). MS (ESI) 350 (M+H).

### EXAMPLE 45

### 3-ethoxy-N-(6-methylpyridin-2-yl)-5-pyrimidin-5-ylpyridin-2-amine

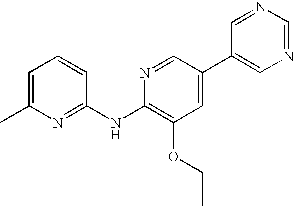

3-Ethoxy-N-(6-methylpyridin-2-yl)-5-pyrimidin-5-ylpyridin-2-amine was obtained by following procedure D using 3-ethoxy-N-(6-methylpyridin-2-yl)-5-(trimethyl stannyl) pyridin-2-amine and 5-bromopyrimidine. ¹H NMR (CDCl₃, 500 MHz) δ 9.19 (s, 1H), 8.96 (s, 2H), 8.40 (d, 1H), 8.09 (s, 1H), 7.92 (s, 1H), 7.60 (t, 1H), 7.22 (s, 1H), 6.81 (d, 1H), 4.21 (q, 2H), 2.46 (s, 3H), 1.56 (t, 3H).

### EXAMPLE 46

### 3-ethoxy-N-(6-methylpyridin-2-yl)-5-[(2-methyl-1,3-thiazol-4-yl)ethynyl]pyridin-2-amine

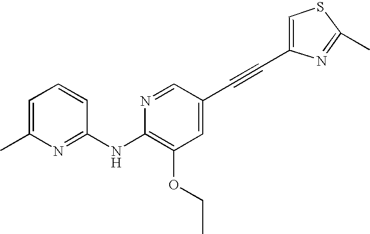

A mixture of 5-bromo-3-ethoxy-N-(6-methylpyridin-2-yl)pyridin-2-amine (100 mg, 0.32 mmol), 2-methyl-4-[(trimethylsilyl)ethynyl]-1,3-thiazole (95 mg, 0.49 mmol), Pd(PPh₃)₄ (37 mg, 0.032 mmol), CuI (12 mg, 0.065 mmol), Et₃N (0.11 mL, 0.81 mmol), and TBAF (1M in THF, 0.5 mL, 0.49 mmol) in THF (5 mL) was placed in a sealed tube and heated to 90° C. for 18 hr. The resulting black mixture was cooled to room temperature, filtered through celite and concentrated. The crude residue was purified by silica gel chromatography (Ethyl acetate/hexanes) to afford 3-ethoxy-N-(6-methylpyridin-2-yl)-5-[(2-methyl-1,3-thiazol-4-yl)ethynyl]pyridin-2-amine. ¹H NMR (CDCl₃, 500 MHz) δ 8.31 (bs, 1H), 8.06 (s, 1H), 7.65 (m, 1H), 7.51 (m, 1H), 7.34 (s, 1H), 7.13 (s, 1H), 6.77 (d, 1H), 4.11 (q, 2H), 2.71 (s, 3H), 2.56 (s, 3H), 1.52 (t, 3H). MS (ESI) 351 (M+H).

### EXAMPLE 47

### 5-chloro-3-ethoxy-N-(6-methylpyridin-2-yl)pyridin-2-amine

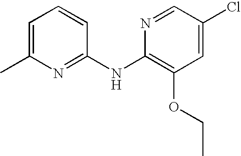

To a solution of 3-hydroxy-5-chloropyridine (5 g, 53 mmol) and Na₂CO₃ (11.4 g, 108 mmol) in H₂O (150 mL) was added I₂ (13.4 g, 53 mmol). After stirring at rt for 1 h, the solution was acidified with conc. HCl to pH˜4 and the resulting precipitate was filtered and washed with H₂O. Recrystallization of the crude residue from 60% aq. EtOH gave 2-iodo-3-hydroxy-5-chloropyridine as a light yellow solid which was used without further purification.

This phenol was alkylated with bromoethane and K₂CO₃ and then coupled with 6-methylpyridin-2-amine following general procedure A to give the title compound as a light yellow solid. ¹H NMR (CDCl₃, 500 MHz) δ 8.30 (d, 1H), 7.77-7.82 (m, 2H), 7.42-7.59 (m, 1H), 6.98 (s, 1H), 6.77 (d, 1H), 4.12 (q, 2H), 2.48 (s, 3H), 1.52 (t, 3H).

### EXAMPLE 48

### 5-chloro-3-ethoxy-2-[(6-methylpyridin-2-yl)oxy]pyridine

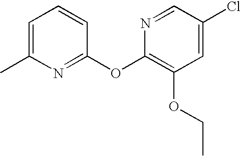

A mixture of 2-iodo-3-hydroxy-5-chloropyridine (300 mg, 1.1 mmol), 2-hydroxy-6-methylpyridine (220 mg, 2.1 mmol) and K₂CO₃ (439 mg, 3.2 mmol) was heated in DMF (4 mL) at 170° C. for 16 h, cooled, and quenched with water. The reaction mixture was extracted with ethyl acetate (4×), dried (MgSO₄), and concentrated. The crude residue was purified by silica gel chromatography (2:1 Et₂O/hex) and then preparative HPLC to give the title compound as a white solid. ¹H NMR (CDCl₃, 500 MHz) δ 7.96 (s, 1H), 7.66 (dt, 1H), 7.28 (s, 1H), 6.88 (d, 1H), 6.69 (d, 1H), 4.10 (q, 2H), 2.45 (s, 3H), 1.38 (t, 3H). MS (ESI) 265 (M⁺).

### EXAMPLE 49

### 5-ethoxy-6-[(6-methylpyridin-2-yl)oxy]-3,3′-bipyridine

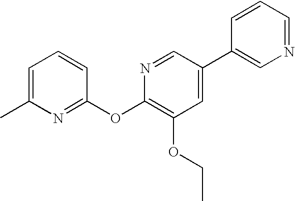

A mixture of 3-(benzyloxy)-5-bromopyridine (1.2 g, 4.6 mmol), 3-pyridylboronic acid (0.73 g, 5.9 mmol), Pd(Ph₃P)₄ (0.53 g, 0.46 mmol) and K₂CO₃ (1.9 g, 13.7 mmol) was warmed to 85° C. in toluene/EtOH/H₂O (15 mL/5 mL/5 mL) for 16 h and then cooled. The reaction was diluted with ethyl acetate and the layers were separated. The aqueous layer was extracted with ethyl acetate (2×) and the combined organic extracts were dried (MgSO₄) and concentrated. Purification by silica gel chromatography (Ethyl acetate) gave 5-(benzyloxy)-3,3′-bipyridine as a colorless solid which was used without further purification.

A mixture of this product (700 mg, 2.7 mmol) and Pd/C in ethyl acetate was stirred under an atmosphere of H₂ at rt until the starting material was consumed as judged by T.L.C. analysis. The reaction mixture was filtered through celite and concentrated. The crude yellow oil (450 mg) was used without further purification.

The crude phenol was iodinated and alkylated with EtBr following the procedure described in Example 47 to give 5-ethoxy-6-iodo-3,3′-bipyridine. MS (ESI) 327 (M+H).

Coupling of 5-ethoxy-6-iodo-3,3′-bipyridine (140 mg, 0.43 mmol) with 2-hydroxy-6-methylpyridine (234 mg, 2.1 mmol) was carried out following the procedure described in Example 48. Preparative HPLC purification gave the title compound as a colorless oil. MS (ESI) 308 (M⁺).

### EXAMPLE 50

### 3-ethoxy-2-[(6-methylpyridin-2-yl)oxy]-5-phenylpyridine

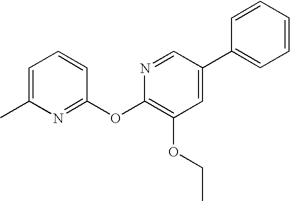

A mixture of the compound from Example 48 (50 mg, 0.19 mmol), phenyl boronic acid (46 mg, 0.38 mmol), Pd₂ dba₃ (17 mg, 0.02 mmol), Cs₂CO₃ (93 mg, 0.29 mmol) and P(t-Bu)₃ (0.08 mL, 0.04 mmol) in toluene (2m) was placed in a sealed tube and heated in a microwave (Personal Chemistry, Model: Smith Creator) for 10 min at 160° C. The reaction mixture was filtered through a celite and concentrated. The residue was purified by preparative HPLC to give the title compound as a pale yellow solid. ¹H NMR (CDCl₃, 500 MHz) δ 8.18-8.20 (m, 1H), 8.10 (d, 1H), 7.96 (dd, 1H), 7.57-7.59 (m, 2H), 7.50-7.54 (m, 2H), 7.44-7.51 (m, 2H), 6.96 (d, 1H), 4.32 (q, 2H), 2.73 (s, 3H), 1.59 (t, 3H). MS (ESI) 306 (M⁺).

### EXAMPLE 51

### N-(6-methylpyridin-2-yl)-3-(2,2,2-trifluoroethoxy)pyridin-2-amine

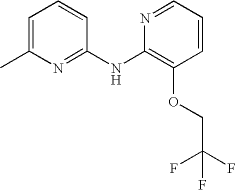

To as solution of 3-hydroxy-2-bromopyridine (0.5 g, 2.87 mmol), trifluoroethanol (2 mL, 28 mmol), and Ph₃P (1.9 g, 7.2 mmol) in THF (15 mL) at 0° C. was added diisopropylazodicarboxylate (DIAD) (10.1 mL, 5.7 mmol). The reaction was allowed to warm to rt overnight. After 16 h, the reaction mixture was concentrated in vacuo and purified by silica gel chromatography (4:1 hexanes/Ethyl acetate) to give 2-bromo-3-trifluoroethoxypyridine as a colorless oil.

The title compound was obtained by following general procedure A using 6-methylpyridin-2-amine and 2-bromo-3-trifluoroethoxypyridine. ¹H NMR (CDCl₃, 500 MHz) δ 8.20 (br s, 1H), 7.91 (d, 1H), 7.59 (br s, 1H), 7.40 (d, 1H), 6.79 (br s, 1H), 6.76 (d, 1H), 6.96 (d, 1H), 4.0-4.1 (m, 1H), 3.99-4.0 (m, 1H), 2.49 (s, 3H).

### EXAMPLE 52

### 3-Ethoxy-N-(6-methylpyridin-2-yl)-5-(pyridin-3-yloxy)pyridin-2-amine

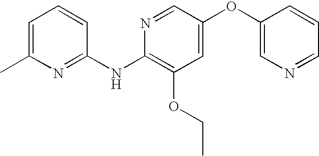

Sodium hydride (2.2 g, 55 mmol) was added in four equal portions to a solution of 5-chloropyridin-3-ol (5.0 g, 39 mmol) and DMF (50 mL) under N₂ at rt. After 15 min, ethyl iodide (3.2 mL, 40 mmol) was added. The resulting mixture was stirred for 16 h at rt and then 30 min at 40° C. The reaction mixture was poured into water (500 mL) and extracted with methyl tert-butyl ether (125 mL×4). The combined organic extracts were dried (MgSO₄), filtered, and concentrated to give 2.7 g of 3-chloro-5-ethoxypyridine. ¹H NMR (CDCl₃, 500 MHz) δ 8.18 (d, 2H), 7.19 (t, 1H), 4.07 (q, 2H), 1.45 (t, 3H).

Nitric acid (2 mL, 32 mmol) was added over 1 min to a solution of 3-chloro-5-ethoxypyridine (2.7 g, 17 mmol) and sulfuric acid (15 mL) at rt. The resulting solution was heated at 60° C. for 1 h, slowly poured into 6 N NaOH (100 mL) at 0° C., and then extracted with chloroform (150 mL×3). The combined organic extracts were dried (MgSO₄), filtered, and concentrated to give 2.8 g of 5-chloro-3-ethoxy-2-nitropyridine. ¹H NMR (CDCl₃, 500 MHz) δ 8.02 (s, 1H), 7.49 (s, 1H), 4.21 (q, 2H), 1.49 (t, 3H).

A mixture of 5-chloro-3-ethoxy-2-nitropyridine (0.43 g, 2.1 mmol), pyridin-3-ol (0.60 g, 6.3 mmol), K₂CO₃ (1.2 g, 8.7 mmol), and DMF (8 mL) were heated at 80° C. under N₂ for 20 min, 100° C. for 2 h, and then 120° C. for 30 min. The resulting mixture was poured into water (125 mL) and extracted with methyl tert-butyl ether (40 mL×4). The combined organic extracts were dried (MgSO₄), filtered, and concentrated to give 3-ethoxy-2-nitro-5-(pyridin-3-yloxy)pyridine.

Hydrazine (51% in H₂O, 0.5 mL, 8.2 mmol) was added over 1 min to a mixture of 3-ethoxy-2-nitro-5-(pyridin-3-yloxy)pyridine (0.28 g, 1.1 mmol), Pd/C (10 wt %, 50 mg, 0.047 mmol Pd), and ethanol (8 mL) at rt. The reaction was heated at reflux for 2 h, filtered through Celite, and then concentrated to give 0.27 g of 3-ethoxy-5-(pyridin-3-yloxy)pyridin-2-amine. ¹H NMR (CDCl₃, 500 MHz) δ 8.38 (s, 1H), 8.31 (d, 1H), 7.53 (s, 1H), 7.24-7.19 (m, 2H), 6.72 (s, 1H), 4.68 (br s, 2H), 4.01 (q, 2H), 1.45 (t, 3H).

A mixture of 3-ethoxy-5-(pyridin-3-yloxy)pyridin-2-amine (0.23 g, 1.0 mmol), 2-bromo-6-methylpyridine (0.20 g, 1.2 mmol), sodium tert-butoxide (0.22 g, 2.3 mmol), Pd₂ dba₃ (30 mg, 0.066 mmol Pd), BINAP (43 mg, 0.069 mmol), and toluene (3 mL) was degassed with bubbling nitrogen for 30 min. The reaction vessel was then sealed and heated in a microwave (Personal Chemistry, Model: Smith Creator) at 130° C. for 15 min. The resulting mixture was purified by silica gel chromatography (ethyl acetate/hexanes) to give 0.11 g of 3-ethoxy-N-(6-methylpyridin-2-yl)-5-(pyridin-3-yloxy)pyridin-2-amine. ¹H NMR (CDCl₃, 500 MHz) δ 8.42 (s, 1H), 8.35-8.32 (m, 1H), 8.31 (d, 1H), 7.76 (br s, 1H), 7.69 (s, 1H), 7.56 (t, 1H) 7.28-7.24 (m, 2H), 6.81 (s, 1H), 6.75 (d, 1H), 4.08 (q, 2H), 2.47 (s, 3H), 1.50 (t, 3H). MS (ESI): 323.5 (M+H).

### EXAMPLE 53

### 3-Ethoxy-N-(6-methylpyridin-2-yl)-5-phenoxypyridin-2-amine

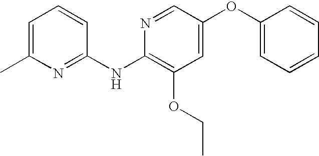

Following example 52, 3-ethoxy-N-(6-methylpyridin-2-yl)-5-phenoxypyridin-2-amine was obtained when phenol was used in place of pyridin-3-ol. ¹H NMR (CDCl₃, 500 MHz) δ 8.32 (d, 1H), 7.73 (br s, 1H). 7.68 (d, 1H), 7.55 (t, 1H), 7.32 (t, 2H), 7.07 (t, 1H), 6.98 (d, 2H), 6.81 (d, 1H), 6.72 (d, 1H), 4.06 (q, 2H), 2.47 (s, 3H), 1.48 (t, 3H). MS (ESI) 322.5 (M+H).

### EXAMPLE 54

### 3-Ethoxy-N-(6-methylpyridin-2.yl)-5-(pyridin-2-yloxy)pyridin-2-amine

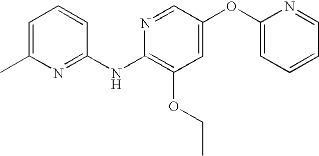

Nitric acid (0.25 mL, 5.0 mmol) was added over 1 min to a solution of 5-fluoropyridin-3-ol (0.25 g, 2.2 mmol) and sulfuric acid (3 mL) at 70° C. After 20 min, a second aliquot (0.25 mL) of nitric acid was added over 1 min. After an additional 40 min, the reaction was cooled to 0° C. Water (25 mL) was added followed by slow addition of 10 N NaOH (13 mL) at 0° C. The solution was neutralized with 1 N HCl and concentrated to give 5-fluoro-2-nitropyridin-3-ol. MS (ESI): 158.8 (M+H).

A mixture of 5-fluoro-2-nitropyridin-3-ol (0.35 g, 2.2 mmol), ethyl iodide (1.4 mL, 18 mmol), K₂CO₃ (3.0 g, 22 mmol), and acetonitrile (40 mL) was heated at 100° C. under N₂. After 5 h, the acetonitrile was removed in vacuo, and the residue was partitioned between water (200 mL) and chloroform (100 mL). The aqueous layer was extracted with chloroform (100 mL×2), and the combined organic extracts were dried (MgSO₄), filtered, and concentrated to give 0.19 g of 3-ethoxy-5-fluoro-2-nitropyridine. ¹H NMR (CDCl₃, 500 MHz) δ 7.93 (dd, 2H), 7.22 (dd, 1H), 4.20 (q, 2H), 1.51 (t, 3H).

A mixture of 3-ethoxy-5-fluoro-2-nitropyridine (69 mg, 0.37 mmol), pyridin-2-ol (0.15 g, 1.6 mmol), Na₂CO₃ (0.35 g, 3.3 mmol), and DMF (2.5 mL) were stirred at rt under N₂ for 15 h and then heated at 50° C. After an additional 10 h, the DMF was removed in vacuo, and the residue was partitioned between water (25 mL) and chloroform (25 mL). The aqueous layer was extracted with chloroform (25 mL×2) and then ethyl acetate (25 mL×2), and the combined organic extracts were dried (MgSO₄), filtered, and concentrated to give 0.13 g of 3-ethoxy-2-nitro-5-(pyridin-2-yloxy)pyridine. MS (ESI): 261.9 (M+H).

3-Ethoxy-2-nitro-5-(pyridin-2-yloxy)pyridine was transformed into 3-ethoxy-N-(6-methylpyridin-2-yl)-5-(pyridin-2-yloxy)pyridin-2-amine according to the last two procedures described in example 52 and then purified by preparative reverse-phase HPLC. ¹H NMR (CD₃OD, 500 MHz) δ 8.17 (t, 1H), 8.11 (s, 1H), 7.73-7.65 (m, 4H), 7.23 (d, 1H), 6.68 (d, 1H), 6.54 (t, 1H) 4.33 (q, 2H), 2.75 (s, 3H), 1.55 (t, 3H). MS (ESI): 323.4 (M+H).

### EXAMPLE 55

### 3-Ethoxy-N-(6-methylpyridin-2-yl)-5-(pyridin-4-yloxy)pyridin-2-amine

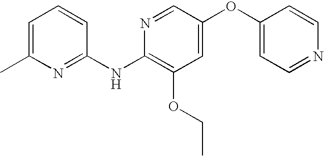

Following example 54, 3-ethoxy-N-(6-methylpyridin-2-yl)-5-(pyridin-4-yloxy)pyridin-2-amine was obtained when pyridin-4-ol was used in place of pyridin-2-ol. ¹H NMR (CDCl₃, 500 MHz) δ 8.33 (d, 1H), 7.90 (br s, 1H). 7.88 (s, 1H), 7.60 (t, 1H), 7.50 (d, 2H), 6.95 (s, 1H), 6.82 (d, 1H), 6.49 (d, 2H), 4.19 (q, 2H), 2.49 (s, 3H), 1.56 (t, 3H). MS (ESI) 323.2 (M+H).

### EXAMPLES 56-199

The compounds described in following examples are synthesized according to the techniques and procedures outlined above. One skilled in the art of organic synthesis would be able to modify these techniques as necessary to achieve the needed compounds.

Examples 56-199 are based on the structure:

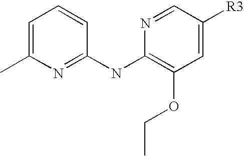

wherein R₃ is selected from table below:

| Example | R₃ |
|---|---|
| 56 | 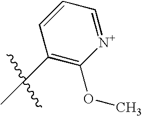 |
| 57 | 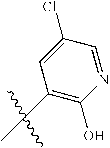 |
| 58 | 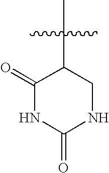 |
| 59 | 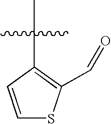 |
| 60 | 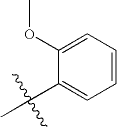 |
| 61 | 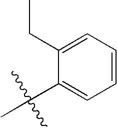 |
| 62 | 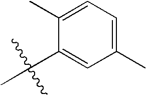 |
| 63 | 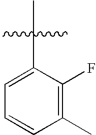 |
| 64 | 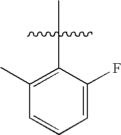 |
| 65 | 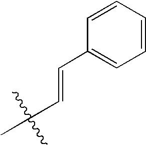 |
| 66 | 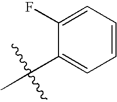 |
| 67 | 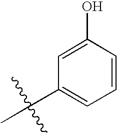 |
| 68 | 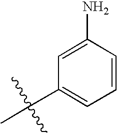 |
| 69 | 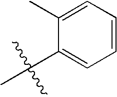 |
| 70 | 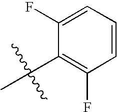 |
| 71 | 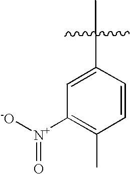 |
| 72 | 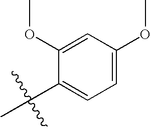 |
| 73 | 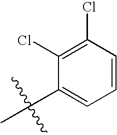 |
| 74 | 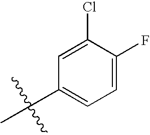 |
| 75 | 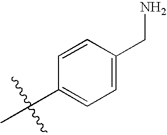 |
| 76 | 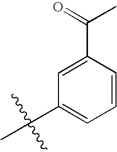 |
| 77 | 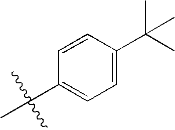 |
| 78 | 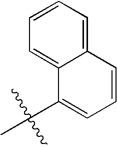 |
| 79 | 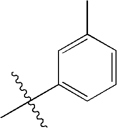 |
| 80 | 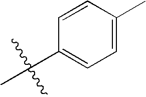 |
| 81 | 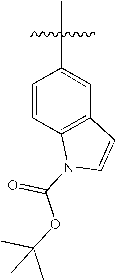 |
| 82 | 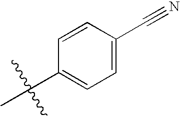 |
| 83 | 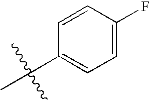 |
| 84 | 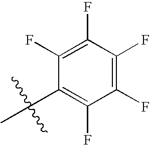 |
| 85 | 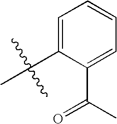 |
| 86 | 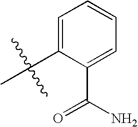 |
| 87 | 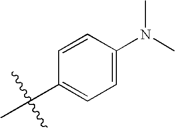 |
| 88 | 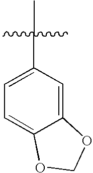 |
| 89 | 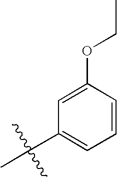 |
| 90 | 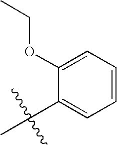 |
| 91 | 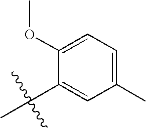 |
| 92 | 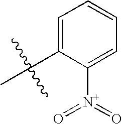 |
| 93 | 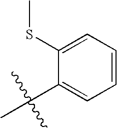 |
| 94 | 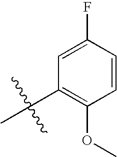 |
| 95 | 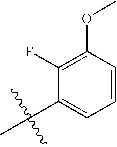 |
| 96 | 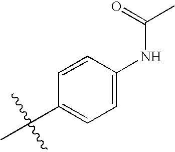 |
| 97 | 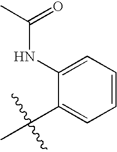 |
| 98 | 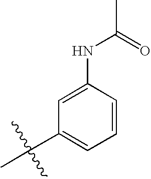 |
| 99 | 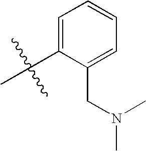 |
| 100 | 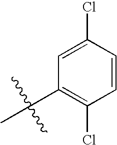 |
| 101 | 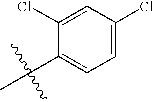 |
| 102 | 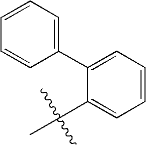 |
| 103 | 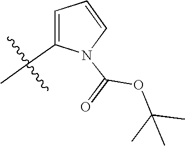 |
| 104 | 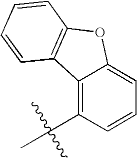 |
| 105 | 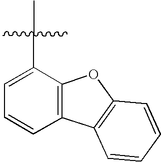 |
| 106 | 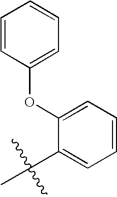 |
| 107 | 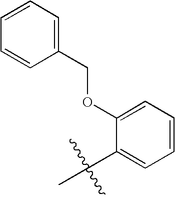 |
| 108 | 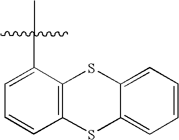 |
| 109 | 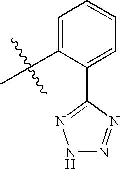 |
| 110 | 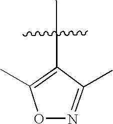 |
| 111 | 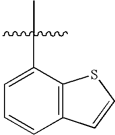 |
| 112 | 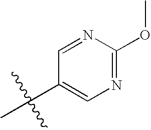 |
| 113 | 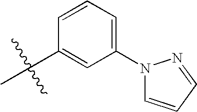 |
| 114 | 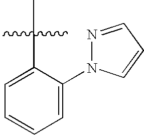 |
| 115 | 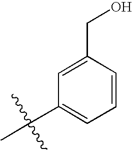 |
| 116 | 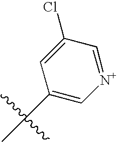 |
| 117 | 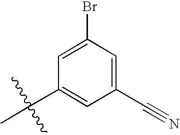 |
| 118 | 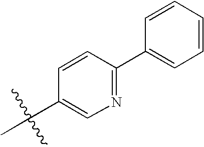 |
| 119 | 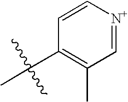 |
| 120 | 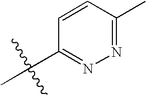 |
| 121 | 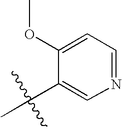 |
| 122 | 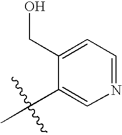 |
| 123 | 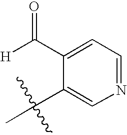 |
| 124 | 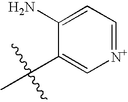 |
| 125 | 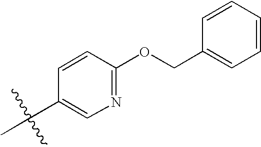 |
| 126 | 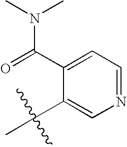 |
| 127 | 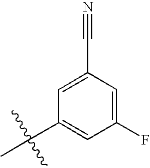 |
| 128 | 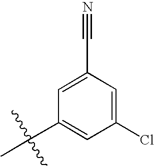 |
| 129 | 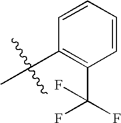 |
| 130 | 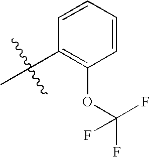 |
| 131 | 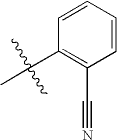 |
| 132 | 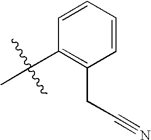 |
| 133 | 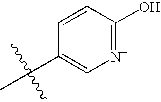 |
| 134 | 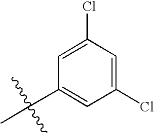 |
| 135 | 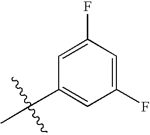 |
| 136 | 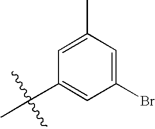 |
| 137 | 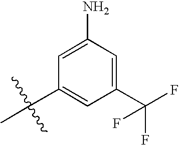 |
| 138 | 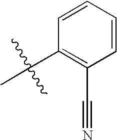 |
| 139 | 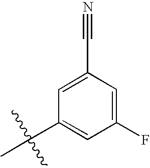 |
| 140 | 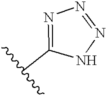 |
| 141 | 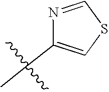 |
| 142 | 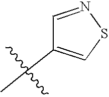 |
| 143 | 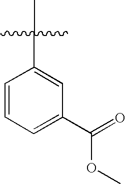 |
| 144 | 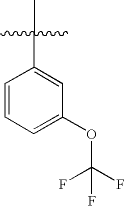 |
| 145 | 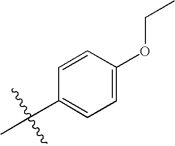 |
| 146 | 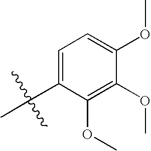 |
| 147 | 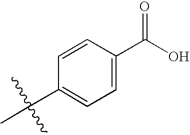 |
| 148 | 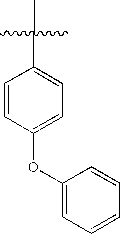 |
| 149 | 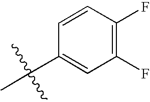 |
| 150 | 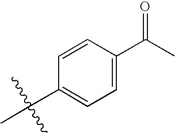 |
| 151 | 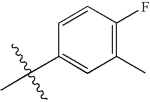 |
| 152 | 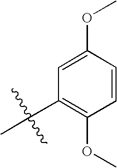 |
| 153 | 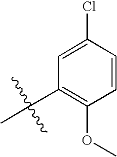 |
| 154 | 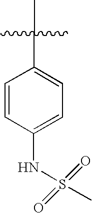 |
| 155 | 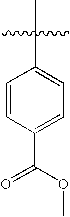 |
| 156 | 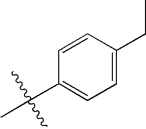 |
| 157 | 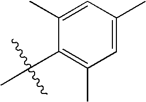 |
| 158 | 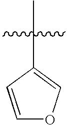 |
| 159 | 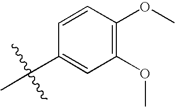 |
| 160 | 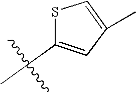 |
| 161 | 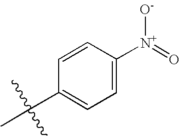 |
| 162 | 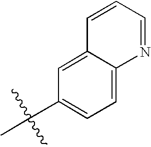 |
| 163 | 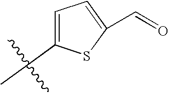 |
| 164 | 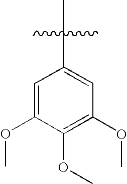 |
| 165 | 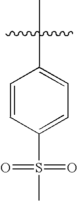 |
| 166 | 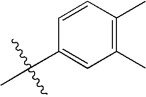 |
| 167 | 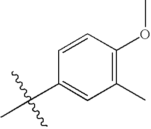 |
| 168 | 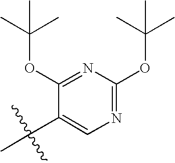 |
| 169 | 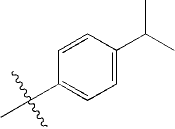 |
| 170 | 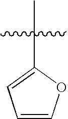 |
| 171 | 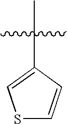 |
| 172 | 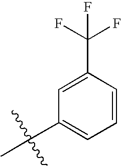 |
| 173 | 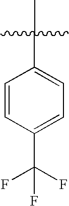 |
| 174 | 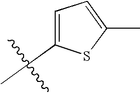 |
| 175 | 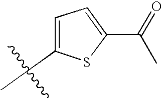 |
| 176 | 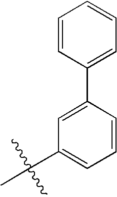 |
| 177 | 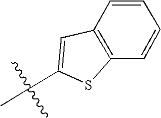 |
| 178 | 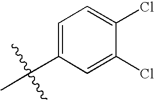 |
| 179 | 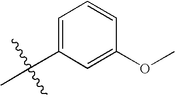 |
| 180 | 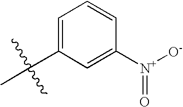 |
| 181 | 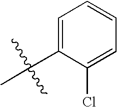 |
| 182 | 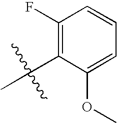 |
| 183 | 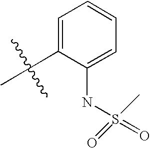 |
| 184 | 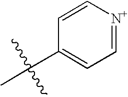 |
| 185 | 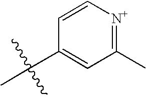 |
| 186 | 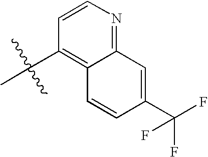 |
| 187 | 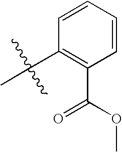 |
| 188 | 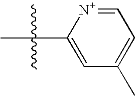 |
| 189 | 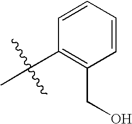 |
| 190 | 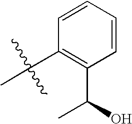 |
| 191 | 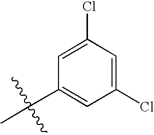 |
| 192 | 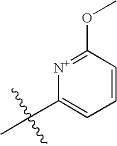 |
| 193 | 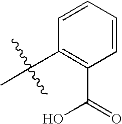 |
| 194 | 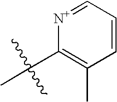 |
| 195 | 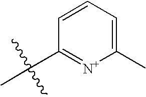 |
| 196 | 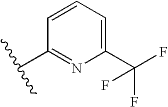 |
| 197 | 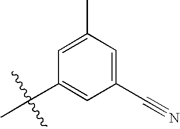 |
| 198 | 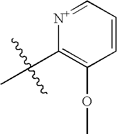 |
| 199 | 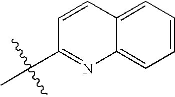 |

### EXAMPLES 200-204

The compounds described in following examples are synthesized according to the techniques and procedures outlined above. One skilled in the art of organic synthesis would be able to modify these techniques as necessary to achieve the needed compounds.

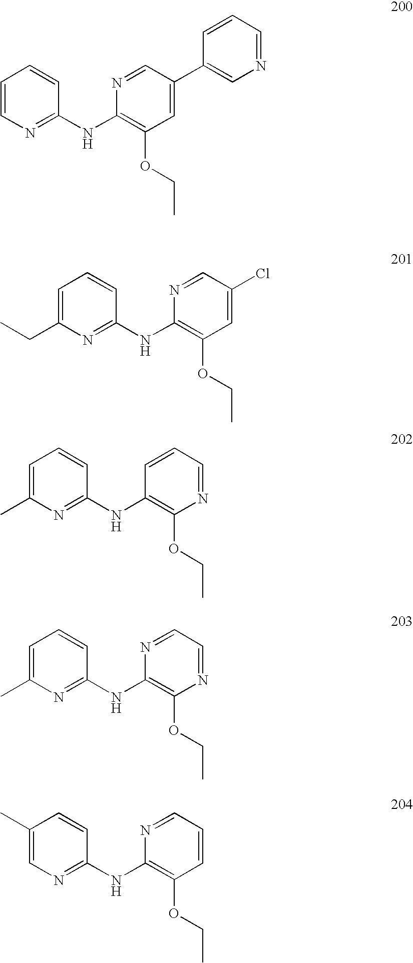

Other variations or modifications, which will be obvious to those skilled in the art, are within the scope and teachings of this invention. This invention is not to be limited except as set forth in the following claims.

What is claimed is:

1. A compound represented by Formula (I): 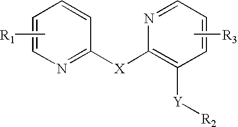
or a pharmaceutically acceptable salt thereof wherein:
R₁ is selected from:
1) hydrogen,
2) C₁₋₁₀alkyl,
3) C₂₋₁₀alkenyl,
4) C₂₋₁₀alkynyl
5) cycloalkyl,
6) heterocyclyl,
7) aryl,
8) heteroaryl,
NR^{d}R^{e},
—CO₂R^{d},
—OR^{d},
—CN, and
halogen,
where alkyl, alkenyl, alkynyl, cycloalkyl and heterocyclyl are optionally substituted with one to four substituents selected from R^{a}, and where aryl and heteroaryl are optionally substituted with one to four substituents independently selected from R^{b};
R₂ is selected from:
1) hydrogen,
2) C₁₋₁₀alkyl,
3) C₂₋₁₀alkenyl,
C₂₋₁₀alkynyl,
cycloalkyl,
heterocyclyl,
aryl, and
heteroaryl,
where alkyl, alkenyl and alkynyl, cycloalkyl and heterocyclyl, aryl, and heteroaryl are optionally substituted with one to four substituents independently selected from R^{b};
R₃ is selected from:
1) R^{b},
2) hydrogen,
-Z-aryl
-Z-heteroaryl
where aryl and heteroaryl are optionally substituted with one to four substituents independently selected from R^{b}, and wherein Z is a bond, C, O, S or NR^{d};
R^{a} is selected from:
1) hydrogen,
2) —OR^{d},
3) —NO₂,
4) halogen,
5) —S(O)ₘR^{d},
6) —SR^{d},
7) —S(O)ₘNR^{d}R^{e},
8) —NR^{d}R^{e},
9) —C(O)R^{d},
10) —CO₂R^{d},
11) —OC(O)R^{d},
12) —CN,
13) —C(O)NR^{d}R^{e},
14) —NR^{d}C(O)R^{e},
15) —OC(O)NR^{d}R^{e},
16) —NR^{d}C(O)OR^{e},
17) —NR^{d}C(O)NR^{d}R^{e},
18) —CR^{d}(N—OR^{d}),
19) CF₃, and
20) —OCF₃;
R^{b} is selected from:
1) a group selected from R^{a},
2) C₁₋₁₀ alkyl,
3) C₂₋₁₀ alkenyl,
4) C₂₋₁₀ alkynyl,
5) cycloalkyl,
6) heterocyclyl,
7) aryl, and
8) heteroaryl,
where alkyl, alkenyl, alkynyl, cycloalkyl, heterocyclyl, aryl, heteroaryl are optionally substituted with one to four substituents selected from a group independently selected from R^{c};
R^{c} is selected from:
1) halogen,
2) amino,
3) carboxy,
4) cyano,
5) C₁₋₄alkyl,
6) C₁₋₄alkoxy,
7) aryl,
8) aryl C₁₋₄alkyl,
9) heteroaryl,
10) hydroxy,
11) CF₃, and
12) aryloxy;
R^{d} and R^{e} are independently selected from hydrogen, C₁₋₁₀alkyl, C₂₋₁₀alkenyl, C₂₋₁₀alkynyl and Cy, where alkyl, alkenyl, alkynyl and Cy are optionally substituted with one to four substituents independently selected from R^{c};
or R^{d} and R^{e} together with the atoms to which they are attached form a ring of 4 to 7 members containing 0-2 additional heteroatoms independently selected from oxygen, sulfur and nitrogen;
Cy is independently selected from cycloalkyl, heterocyclyl, aryl, or heteroaryl;
m is 1 or 2;
X is —NR^{d}—, —O—, or —S—;
Y is a bond, —O—, —NR^{a}—or —S—.

2. The compound according to claim 1, or a pharmaceutically acceptable salt thereof, wherein:
R₁ is C₁₋₁₀alkyl, optionally substituted with one to four substituents selected from R^{a},
R₂ is C₁₋₁₀alkyl, optionally substituted with one to four substituents independently selected from R^{b};
X is —NR^{d}—; and
Y is —O—.

3. A compound selected from the following table: 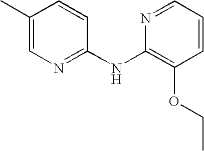 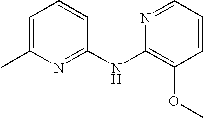 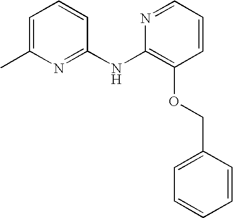 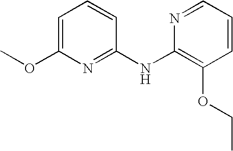 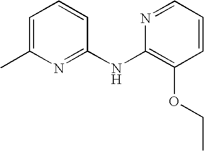 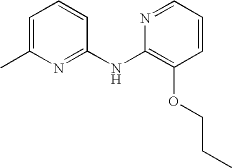 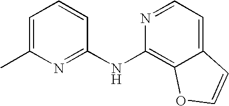 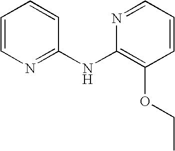 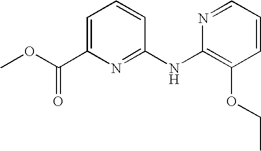 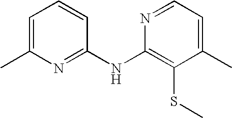 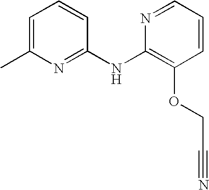 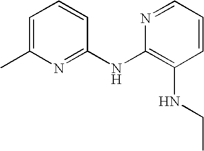 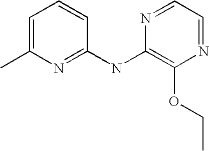 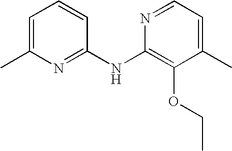 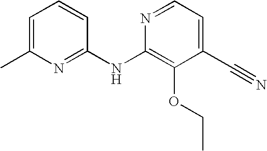 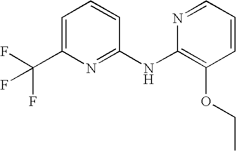 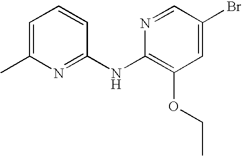 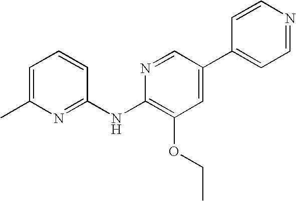 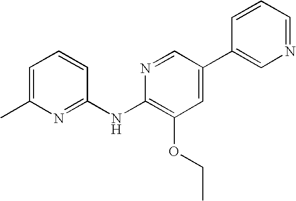 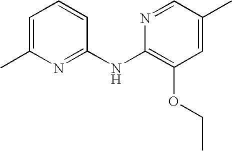 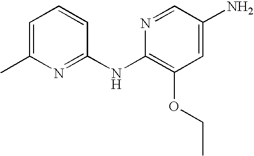 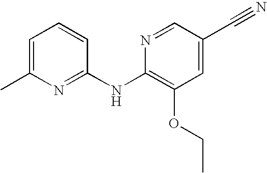 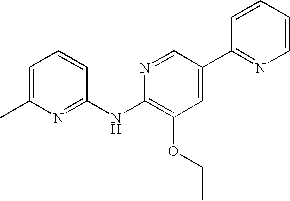 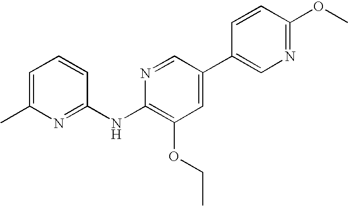 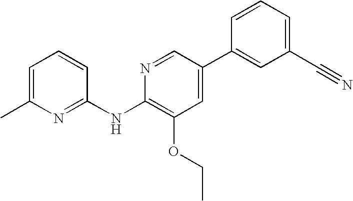 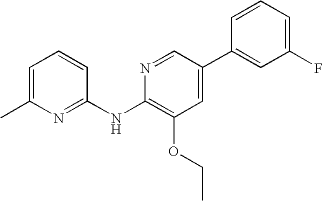 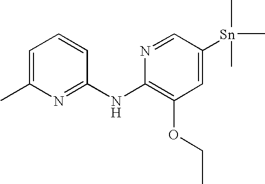 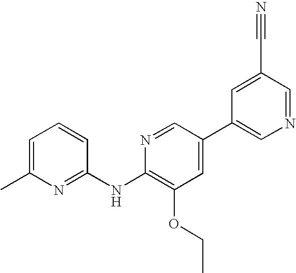 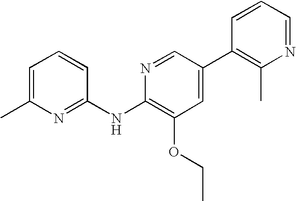 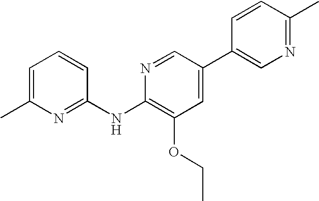 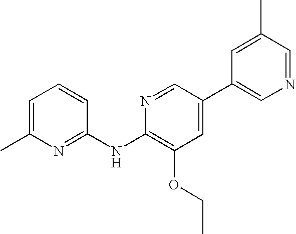 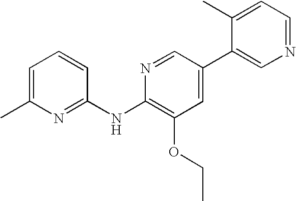 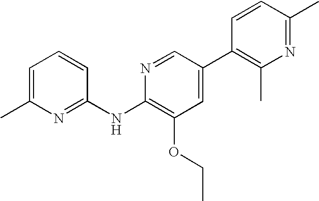 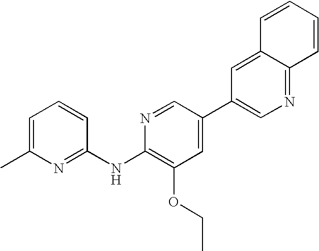 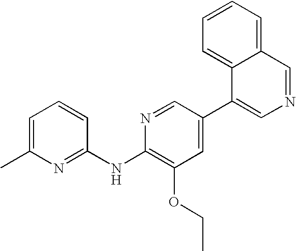 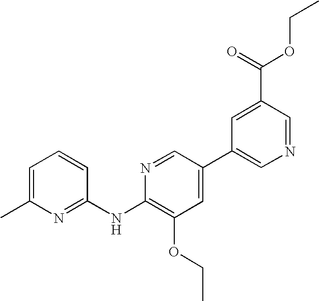 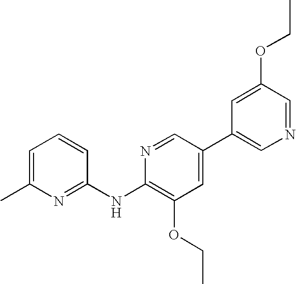 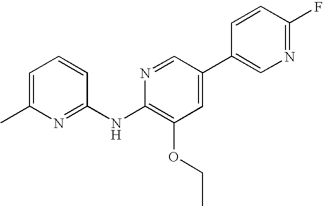 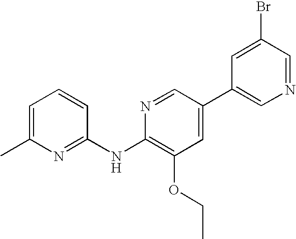 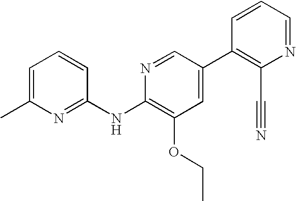 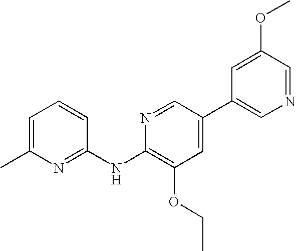 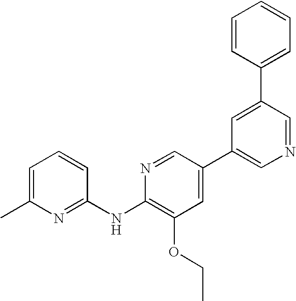 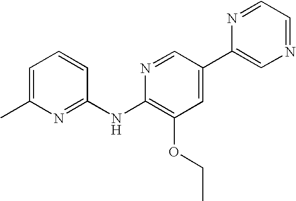 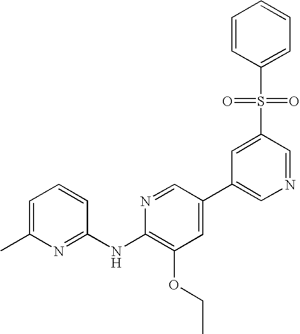 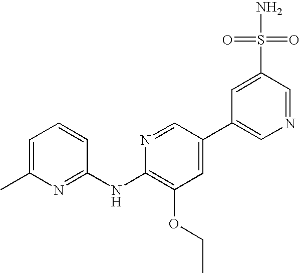 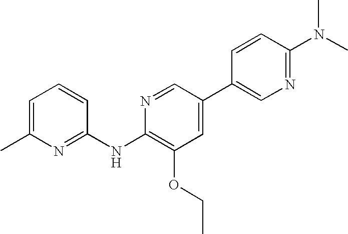 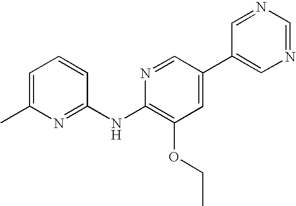 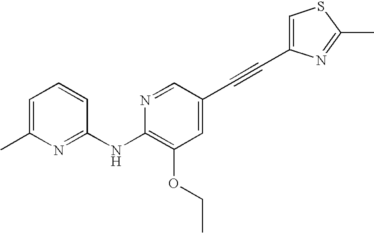 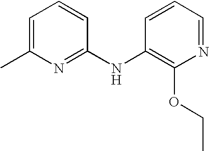 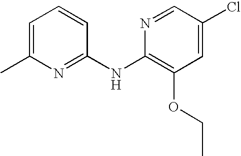 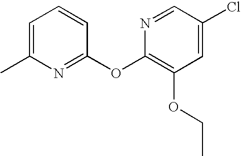 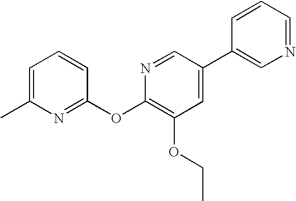 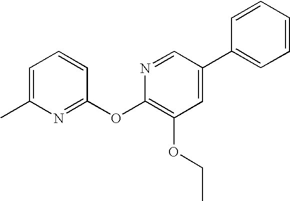 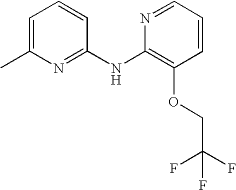 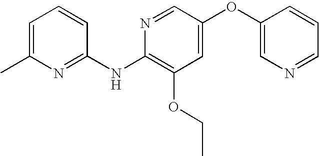 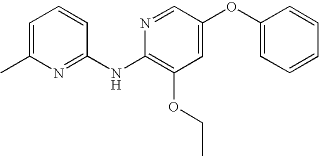 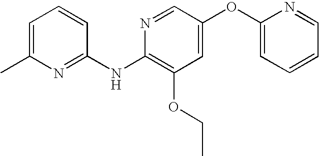 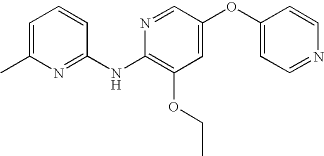 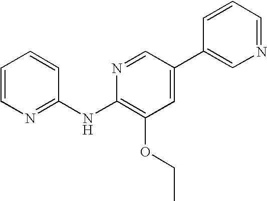 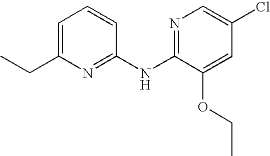
or a pharmaceutically acceptable salt thereof.

4. A compound represented by the formula: 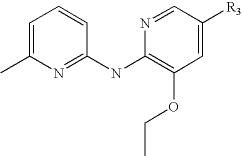
or a pharmaceutically acceptable salt thereof, wherein R₃ is selected from:

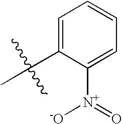

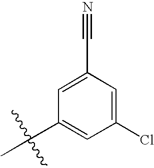

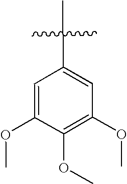

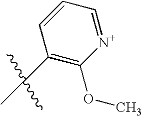

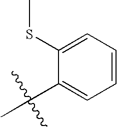

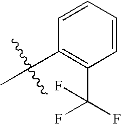

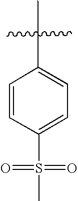

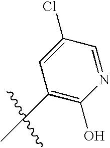

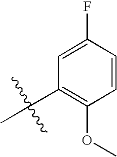

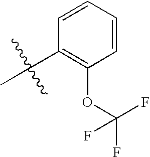

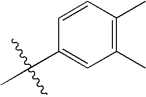

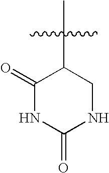

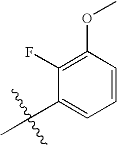

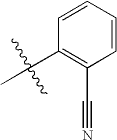

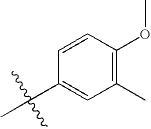

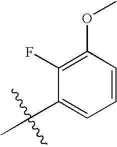

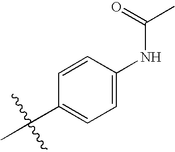

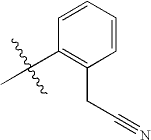

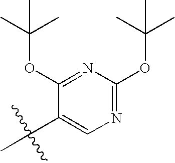

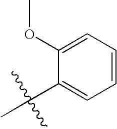

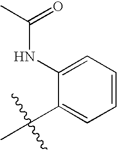

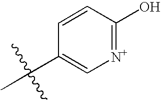

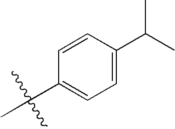

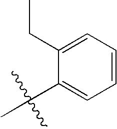

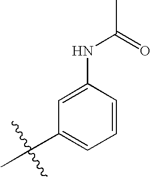

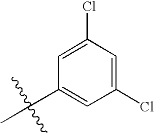

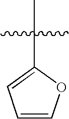

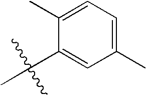

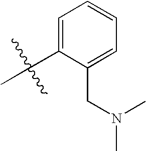

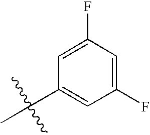

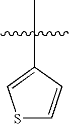

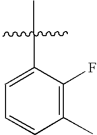

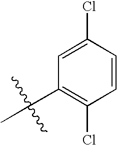

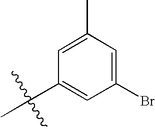

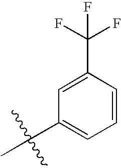

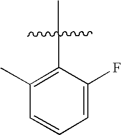

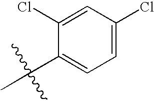

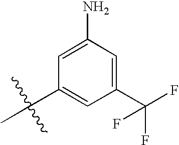

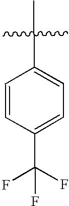

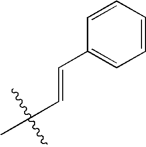

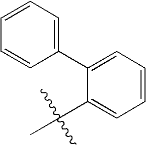

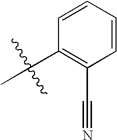

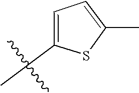

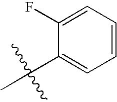

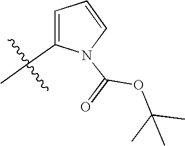

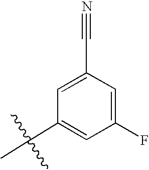

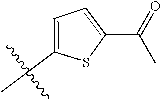

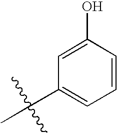

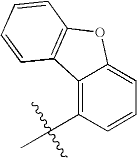

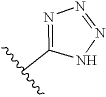

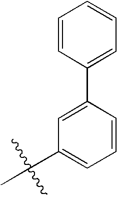

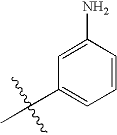

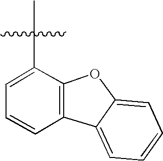

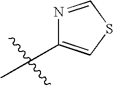

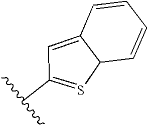

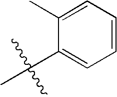

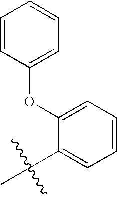

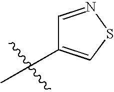

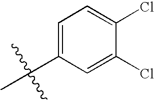

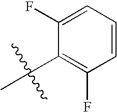

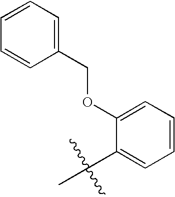

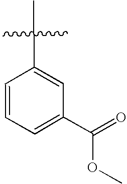

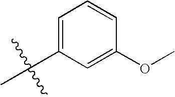

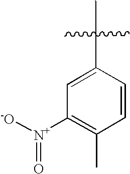

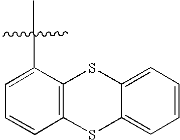

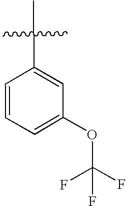

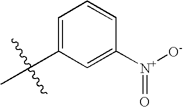

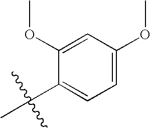

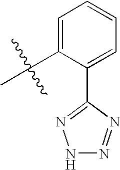

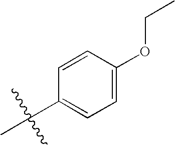

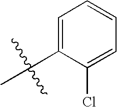

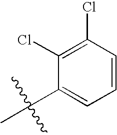

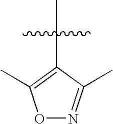

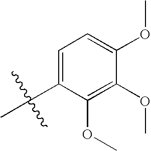

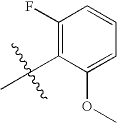

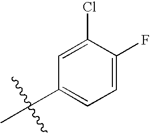

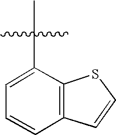

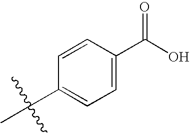

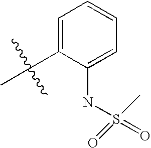

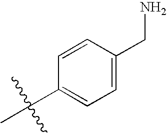

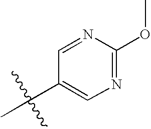

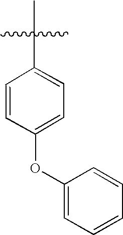

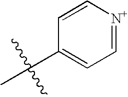

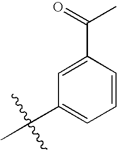

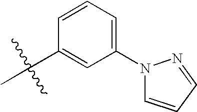

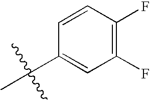

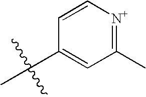

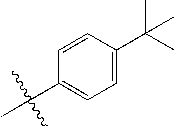

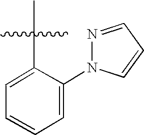

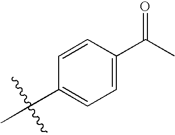

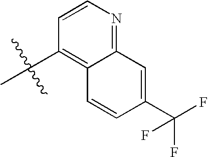

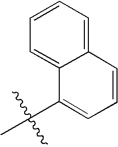

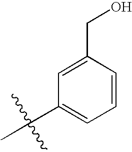

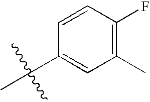

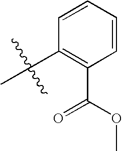

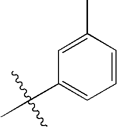

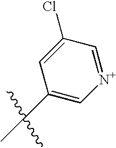

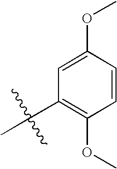

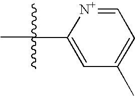

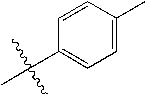

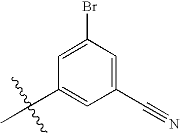

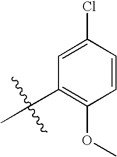

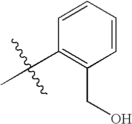

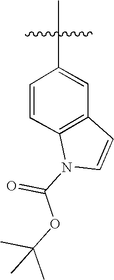

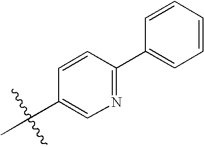

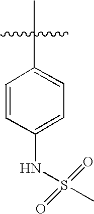

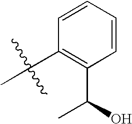

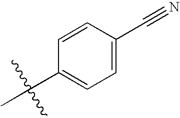

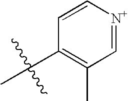

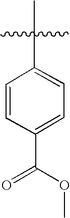

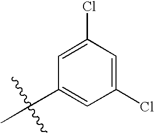

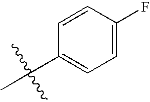

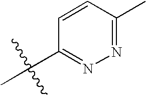

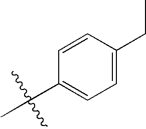

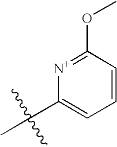

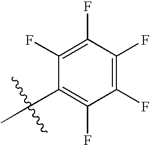

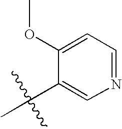

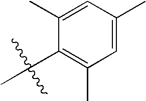

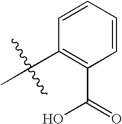

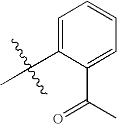

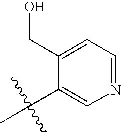

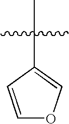

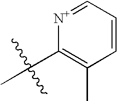

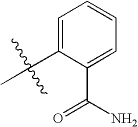

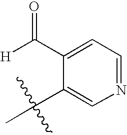

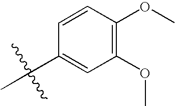

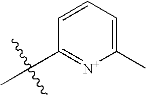

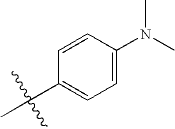

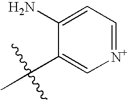

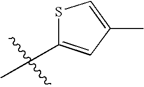

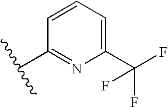

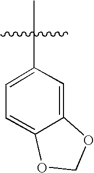

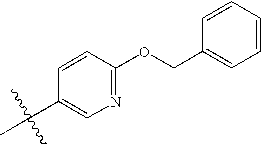

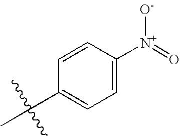

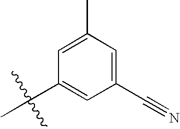

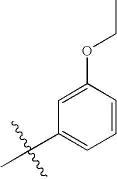

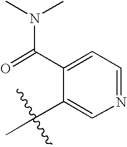

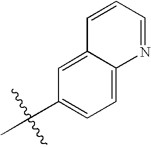

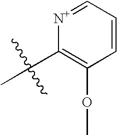

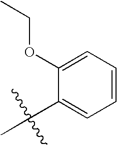

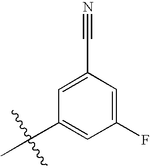

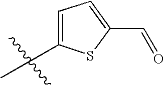

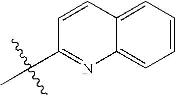

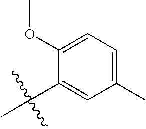

5. A pharmaceutical composition comprising a therapeutically effective amount of the compound according to claim 1, or a pharmaceutically acceptable salt thereof, and a pharmaceutically acceptable carrier.

6. A pharmaceutical composition comprising a therapeutically effective amount of the compound according to claim 2, or a pharmaceutically acceptable salt thereof, and a pharmaceutically acceptable carrier.

7. A pharmaceutical composition comprising a therapeutically effective amount of the compound according to claim 3, or a pharmaceutically acceptable salt thereof, and a pharmaceutically acceptable carrier.

8. A pharmaceutical composition comprising a therapeutically effective amount of the compound according to claim 4, or a pharmaceutically acceptable salt thereof, and a pharmaceutically acceptable carrier.

9. A method of treatment or prevention of pain comprising the step of administering a therapeutically effective amount, or a prophylactically effective amount, of the compound according to claim 1 or a pharmaceutically acceptable salt thereof.

10. A method of treatment or prevention of a pain disorder wherein said pain disorder is acute pain, persistent pain, chronic pain, inflammatory pain, or neuropathic pain, comprising the step of administering a therapeutically effective amount, or a prophylactically effective amount, of the compound according to claim 1 or a pharmaceutically acceptable salt thereof.

11. A method of treatment or prevention of anxiety, depression, bipolar disorder, psychosis, drug withdrawal, tobacco withdrawal, memory loss, cognitive impairment, dementia, Alzheimer's disease, schizophrenia or panic comprising the step of administering a therapeutically effective amount, or a prophylactically effective amount, of the compound according to claim 1 or a pharmaceutically acceptable salt thereof.

12. A method of treatment or prevention of Parkinson's disease comprising the step of administering a therapeutically effective amount, or a prophylactically effective amount, of the compound according to claim 1 or a pharmaceutically acceptable salt thereof.

13. A method of treatment or prevention of anxiety disorders comprising the step of administering a therapeutically effective amount, or a prophylactically effective amount, of the compound according to claim 1 or a pharmaceutically acceptable salt thereof.

14. The method of claim 13 wherein said anxiety disorder is panic attack, agoraphobia or specific phobias, obsessive-compulsive disorders, post-traumatic stress disorder, acute stress disorder, generalized anxiety disorder, eating disorder, substance-induced anxiety disorder, or nonspecified anxiety disorder.

15. A method of treatment or prevention of neuropathic pain comprising the step of administering a therapeutically effective amount, or a prophylactically effective amount, of the compound according to claim 1 or a pharmaceutically acceptable salt thereof.

16. A method of treatment or prevention of depression comprising the step of administering a therapeutically effective amount, or a prophylactically effective amount, of the compound according to claim 1 or a pharmaceutically acceptable salt thereof.

17. A method of treatment or prevention of epilepsy comprising the step of administering a therapeutically effective amount, or a prophylactically effective amount, of the compound according to claim 1 or a pharmaceutically acceptable salt thereof.

18. A method of treatment or prevention of inflammatory pain comprising the step of administering a therapeutically effective amount, or a prophylactically effective amount, of the compound according to claim 1 or a pharmaceutically acceptable salt thereof.

19. A method of treatment or prevention of cognitive dysfunction comprising the step of administering a therapeutically effective amount, or a prophylactically effective amount, of the compound according to claim 1 or a pharmaceutically acceptable salt thereof.

20. A method of treatment or prevention of drug addiction, drug abuse and drug withdrawal comprising the step of administering a therapeutically effective amount, or a prophylactically effective amount, of the compound according to claim 1 or a pharmaceutically acceptable salt thereof.

21. A method of treatment or prevention of bipolar disorders comprising the step of administering a therapeutically effective amount, or a prophylactically effective amount, of the compound according to claim 1 or a pharmaceutically acceptable salt thereof.

22. A method of treatment or prevention of circadian rhythm and sleep disorders comprising the step of administering a therapeutically effective amount, or a prophylactically effective amount, of the compound according to claim 1 or a pharmaceutically acceptable salt thereof.

23. The method of claim 22 wherein the circadian rhythm and sleep disorders are shift-work induced sleep disorder or jet-lag.

24. A method of treatment or prevention of obesity comprising the step of administering a therapeutically effective amount, or a prophylactically effective amount, of the compound according to claim 1 or a pharmaceutically acceptable salt thereof.
